(19) United States
(12) Patent Application Publication
(10) Pub. No.: US 2023/0240516 A1
(43) Pub. Date: Aug. 3, 2023

(54) ENDOSCOPE AND ENDOSCOPE SYSTEM

(71) Applicant: OLYMPUS CORPORATION, Tokyo (JP)

(72) Inventor: Azusa NOGUCHI, Tokyo (JP)

(73) Assignee: OLYMPUS CORPORATION, Tokyo (JP)

(21) Appl. No.: 18/133,537

(22) Filed: Apr. 12, 2023

Related U.S. Application Data
(63) Continuation of application No. PCT/JP2021/006633, filed on Feb. 22, 2021.

(51) Int. Cl. A61B 1/00; A61B 1/07; A61B 1/05

(52) U.S. Cl. CPC A61B 1/00137; A61B 1/05; A61B 1/07; A61B 1/00009

(57) ABSTRACT

An endoscope includes an insert part. The endoscope has an imaging unit provided in the insert part, a light guide having a light guide exit surface from which illumination light is emitted provided in the insert part and an end cover provided at the distal end of the insert part. The end cover has a cut-through portion in which the imaging unit is inserted and fixed. The end cover has a first surface on its object side, a second surface on its operator side, and a side surface on its outer circumference. The first surface includes a first flat surface and a curved surface. The curved surface is located between the first flat surface and the side surface. The second surface has an illumination area on which the illumination light is incident. At least a portion of the illumination area is formed as a partial torus surface. First specific cross sections are defined as cross sections containing the center axis of the end cover and intersecting the light guide exit surface. The endoscope satisfies the following conditional expression (1) in at least one first specific cross section:

$$0 \leq \text{hA} / \text{r} \leq 0.5$$

### CROSS REFERENCES TO RELATED APPLICATION

The present application is a continuation application of PCT/JP2021/006633 filed on Feb. 22, 2021; the entire contents of which are incorporated herein by reference.

### BACKGROUND OF INVENTION

### Technical Field

The present invention relates to an endoscope and an endoscope system.

### Description of the Related Art

An endoscope having an end cover is disclosed in Japanese Patent Application Laid-Open No. 2008-237790. The end cover is attached to the distal end of the insert part of the endoscope. The end cover is made of a transparent material.

In the end portion of the insert part are provided an objective optical system, light guides, an imager, and the end cover. The light guides are disposed on two sides of the objective optical system. The imager has an image output area i.e., the area within which the subject image can be acquired from the imager.

The end cover has a first surface, a second surface and a side surface. The first surface is located on the object side. The second surface is located on the near side or the operator side. The side surface is located on the outer circumference.

The first side is made up of a flat surface and a curved surface. The curved surface extends between the flat surface and the side surface. The curved surface provided as above allows the insert part to be inserted easily into a human body.

The second surface includes a portion opposed to the exit surface of the light guide. This portion constitutes an illumination light diffuser. The illumination light emitted from the exit surface of the light guide passes through the illumination light diffuser and goes out from the first surface of the end cover to illuminate the field of view.

The field of view refers to an area in the object space that can be seen through an optical device. The field of view of an endoscope is determined by the focal length and distortion of the objective optical system and the image output area of the imager.

The illumination light emitted from the illumination light diffuser enters the flat surface and the curved surface in the first surface. The curved surface in the first surface has a positive refractive power. Hence, the illumination light incident on the curved surface is refracted in directions toward the center of the field of view. The illumination light refracted in directions toward the center of the field of view overlaps, on the object, with the illumination light going out from the flat surface. In consequence, the intensity of the illumination light is high at specific positions in the field of view, resulting in uneven illumination.

### SUMMARY

An endoscope according to at least some of embodiments comprises:
an insert part;
an imaging unit provided in the insert part;
a light guide having a light guide exit surface provided in the insert part; and
an end cover provided at the distal end of the insert part, wherein
illumination light is emitted from the light guide exit surface,
the end cover has a cut-through portion in which the imaging unit is inserted and fixed,
the end cover has a first surface on its object side, a second surface on its operator side, and a side surface on its outer circumference,
the first surface includes a first flat surface and a curved surface,
the curved surface is located between the first flat surface and the side surface,
the second surface has an illumination area on which the illumination light is incident,
at least a portion of the illumination area is formed as a partial torus surface,
first specific cross sections are defined as cross sections containing the center axis of the end cover and intersecting the light guide exit surface, and
the endoscope satisfies the following conditional expression (1) in at least one first specific cross section:
$$0 \leq \text{hA} / \text{r} \leq 0.5$$
where the partial torus surface is defined as a surface partially cut out from a torus surface, the torus surface is the surface of a body of revolution formed by rotating a circle in a plane about an axis of rotation that lies in the same plane and does not intersect the circle, the circle is referred to as a small circle, hA is the distance between a first intersection point and a second intersection point, the first intersection point is the point of intersection of the straight line passing through the center of the small circle and parallel to the center axis and the light guide exit surface, the second intersection point is the point of intersection of a ray passing through the boundary of the first flat surface and the curved surface and parallel to the center axis in the space between the light guide exit surface and the illumination area and the light guide exit surface, in the case where the second intersection point is located between the first intersection point and the side surface, the distance is represented by a value with a plus sign, and r is the radius of the small circle.

An endoscope according to at least some of embodiments comprises:
an insert part;
an imaging unit provided in the insert part;
a light guide having a light guide exit surface provided in the insert part; and
an end cover provided at the distal end of the insert part, wherein
illumination light is emitted from the light guide exit surface,
the end cover has a cut-through portion in which the imaging unit is inserted and fixed,
the end cover has a first surface on its object side, a second surface on its operator side, and a side surface on its outer circumference,
the first surface includes a first flat surface and a curved surface,
the curved surface is located between the first flat surface and the side surface,
the second surface has an illumination area on which the illumination light is incident,
at least a portion of the illumination area is formed as a partial torus surface,
first specific cross sections are defined as cross sections containing the center axis of the end cover and intersecting the light guide exit surface, and
the endoscope satisfies the following conditional expression (2) in at least one first specific cross section: section:
$$0 \leq \left(\text{hA} \times \text{n2}\right) / \text{r} \leq 1.36$$
where a partial torus surface is defined as a surface partially cut out from a torus surface, the torus surface is the surface of a body of revolution formed by rotating a circle in a plane about an axis of rotation that lies in the same plane and does not intersect the circle, this circle is referred to as a small circle, hA is the distance between a first intersection point and a second intersection point, the first intersection point is the point of intersection of the straight line passing through the center of the small circle and parallel to the center axis and the light guide exit surface, the second intersection point is the point of intersection of a ray passing through the boundary of the first flat surface and the curved surface and parallel to the center axis in the space between the light guide exit surface and the illumination area and the light guide exit surface, in the case where the second intersection point is located between the first intersection point and the side surface, the distance is represented by a value with a plus sign, n is the refractive index of the material of the end cover for the e-line, and r is the radius of the small circle.

An endoscope system according to at least some of embodiments comprises one of the endoscopes described above and an image processing apparatus.

### BRIEF DESCRIPTION OF THE DRAWINGS

FIG. 1 is a diagram illustrating an endoscope and an endoscope system according to an embodiment;

FIGS. 2A and 2B are diagrams illustrating a first example of an endoscope having a basic structure;

FIGS. 3A, 3B, and 3C are diagrams illustrating a torus surface;

FIGS. 4A and 4B are diagrams illustrating a second example of the endoscope having the basis structure;

FIGS. 5A, 5B, 5C, and 5D are diagrams showing the end portion of endoscopes as seen from the object side;

FIG. 6 is a cross sectional view of an end cover;

FIGS. 7A and 7B are diagrams showing the end portion of endoscopes as seen from the object side;

FIG. 8 is a cross sectional view of an end cover;

FIG. 9 is a cross sectional view of an end cover;

FIG. 10 is a cross sectional view of an end cover;

FIG. 11 is a diagram showing the end portion of an endoscope as seen from the object side;

FIGS. 12A and 12B are diagrams illustrating the end portion of an insert part and an end cover;

FIGS. 13A and 13B are cross sectional views of end covers;

FIG. 14 is a diagram showing the end portion of an endoscope as seen from the object side;

FIGS. 15A, 15B, 15C, 15D show a first example of the endoscope and the distribution of illumination light thereof;

FIGS. 16A, 16B, 16C, 16D show a second example of the endoscope and the distribution of illumination light thereof;

FIGS. 17A, 17B, 17C, 17D show a third example of the endoscope and the distribution of illumination light thereof;

FIGS. 18A, 18B, 18C, 18D show a fourth example of the endoscope and the distribution of illumination light thereof;

FIGS. 19A, 19B, 19C, 19D show a fifth example of the endoscope and the distribution of illumination light thereof;

FIGS. 20A, 20B, 20C, 20D show a sixth example of the endoscope and the distribution of illumination light thereof;

FIGS. 21A, 21B, 21C, 21D show a seventh example of the endoscope and the distribution of illumination light thereof;

FIGS. 22A, 22B, 22C, 22D show an eighth example of the endoscope and the distribution of illumination light thereof.

### DETAILED DESCRIPTION

Endoscopes according to some embodiments and endoscope systems according to some embodiments will be described below with respect to the reason why they are configured as described and their operation. It should be understood that the present invention is not limited by the embodiments.

In the following description, reference will be made to cross sectional views of endoscopes and views of the end portion of the endoscopes seen from the object side. Each cross sectional view shows a cross section in the plane containing the center axis of the end cover.

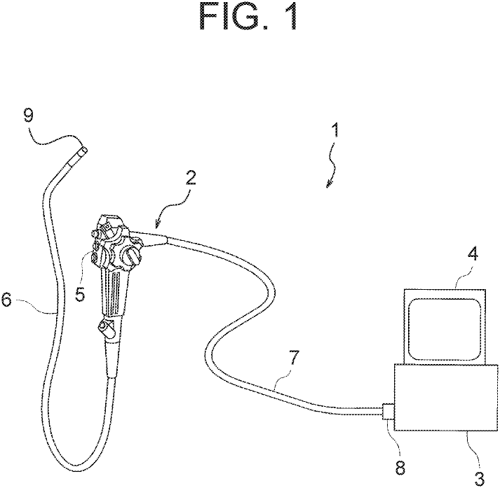

FIG. 1 is a diagram illustrating an endoscope and an endoscope system according to one embodiment. The endoscope may be an electronic endoscope.

The endoscope system 1 shown in FIG. 1 is an observation system using an electronic endoscope 2. The endoscope system 1 includes the electronic endoscope 2, a camera control unit (CCU), and a cabinet 3 having a power supply function. The cabinet 3 is connected with a display unit 4.

The electronic endoscope 2 has an insert part 6, an operation unit 5, a universal cord 7, and a connector 8. The insert part 6 is a thin, long part that can be inserted into the body cavity of a patient. The insert part 6 is made as a flexible part. The observer can conduct various operations using an angle knob and other parts provided in the operation unit 5. The universal cord 7 is connected to the cabinet 3 with the connector 8.

The universal cord 7 contains a signal cable used to transmit various signals and a light guide cable in it. Examples of the transmitted signals include a power voltage signal and a CCD drive signal. These signals are transmitted from the cabinet 3 to the electronic endoscope 2. Another example of the signals is a video signal. The video signal is transmitted from the electronic endoscope 2 to the cabinet 3.

In the cabinet 3 is provided a video processor, which can be connected to a peripheral device, such as a VTR deck or a video printer (not shown in the drawings) . The video processor applies signal processing on the video signal transmitted from the electronic endoscope 2. An endoscope image based on the video signal is displayed on the display screen of the display unit 4.

The distal end of the insert part of the electronic endoscope 2 is provided with an end cover 9.

The endoscope according to this embodiment will be described using first and second examples of the endoscope. The first and second examples of the endoscope has a basic structure.

The basic structure includes an imaging unit and a light guide provided in the insert part. The distal end of the insert part is provided with the end cover. Illumination light is emitted from the exit surface of the light guide, i.e., the light guide exit surface. The end cover has a cut-through portion (or a through bore) through which the imaging unit is inserted and fixed. The object side surface of the end cover will be referred to as the first surface, the surface on the operator side will be referred to as the second surface, and the outer circumferential surface will be referred to as the side surface. The first surface includes a first flat surface and a curved surface. The curved surface extends between the first flat surface and the side surface. The second surface includes an illumination area throughout which illumination light enters. At least a portion of the illumination area has the shape of a partial torus (or torus) surface. In this disclosure, the first specific cross section is defined as a cross section that contains the center axis of the end cover and intersects with the exit surface of the light guide.

The endoscope includes, for example, the operation unit and the insert part. One distal end of the insert part is located on the object side, and the other end is located on the operation unit side. The operation unit is operated by the operator for various operations. The operation unit is disposed at hand of the operator. The term “operator side” will be used to refer to the side near the operation unit.

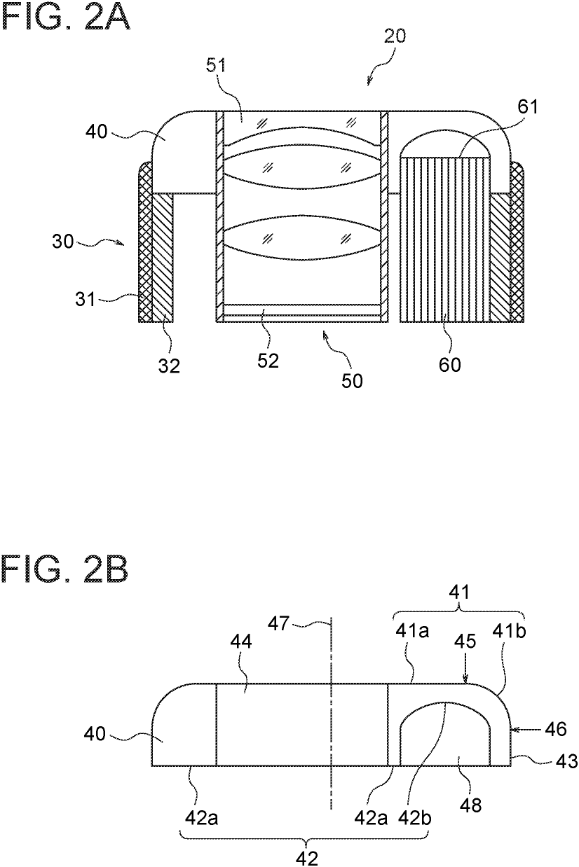

FIGS. 2A and 2B illustrate the first example of the endoscope according to the first embodiment. FIG. 2A is a cross sectional view of the end portion of the insert part. FIG. 2B is a cross sectional view of the end cover.

As illustrated in FIG. 2A, the end portion 30 of the insert part of the endoscope 20 is provided with an end cover 40. The end portion 30 of the insert part includes, for example, a sheathing tube 31 and a metal pipe 32. The sheathing tube 31 and the metal pipe 32 are in contact with the end cover 40.

In the end portion 30 of the insert part are disposed an imaging unit 50 and a light guide 60. The imaging unit 50 includes an objective optical system 51 and an imager 52. The light guide 60 has a light guide exit surface 61. The illumination light is emitted from the light guide exit surface 61.

The illumination light emitted from the light guide exit surface 61 enters the end cover 40. The end cover 40 is made of a transparent material. The transparent material used may be a resin. The illumination light incident on the end cover 40 is refracted and transmitted by the end cover 40 and then goes out from the end cover 40.

The light guide 60 is disposed side by side with and in parallel to the imaging unit 50 in the endoscope 20. Thus, the illumination light is cast in one direction toward the object.

As illustrated in FIG. 2B, the end cover 40 has a first surface 41, a second surface 42, and a side surface 43. The first surface 41 is the object side surface of the end cover 40. The second surface 42 is the operator side surface of the end cover 40. The side surface 43 is the outer circumferential surface of the end cover 40.

The end cover 40 has a through bore 44. The imaging unit 50 is inserted in and fixed to the through bore 44.

The first surface 41 includes a first flat surface 41a and a curved surface 41b. The curved surface 41b extends between the first flat surface 41a and the side surface 43.

The first flat surface 41a, the curved surface 41b, and the side surface 43 are arranged in such a way that they are smoothly connected to each other at their boundaries . The arrow 45 in FIG. 2B indicates the location of the boundary of the first surface 41a and the curved surface 41b. The arrow 46 in FIG. 2B indicates the location of the boundary of the curved surface 41b and the side surface 43.

FIG. 2B shows the center axis 47 of the end cover 40. The center of the through bore 44 is not located on the center axis 47.

The second surface 42 of the end cover 40 includes non-illumination areas 42a and an illumination area 42b. The non-illumination areas 42a are flat surfaces.

The end cover 40 has a recess 48 located at a position opposed to the light guide 60. The illumination area 42b is constituted by the bottom surface of the recess 48. The illumination light emitted from the light guide exit surface 61 is incident on the illumination area 42b.

Since the end cover 40 is made of a transparent material, the illumination light incident on the illumination area 42b is refracted and transmitted through it and goes out from the first surface 41.

In the case of the first example of the basic structure, at least a portion of the illumination area has the shape of a partial torus surface (which will be hereinafter referred to as PT “surface”) . The entire surface of the illumination area 42b in the endoscope 20 is a PT surface.

The PT surface is a surface partially cut out a torus surface. The torus surface is the surface of a body of revolution formed by rotating a circle in a plane about an axis of rotation that lies in the same plane and does not intersect the circle. This circle will be referred to as the “small circle”.

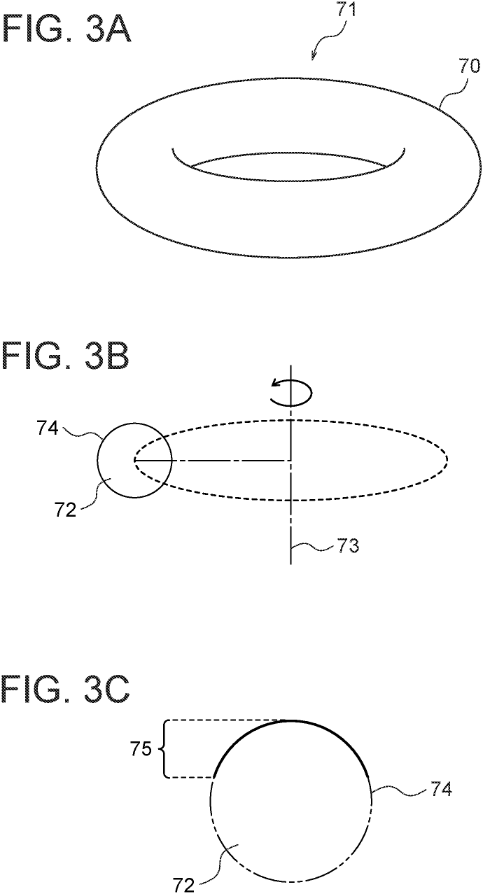

FIGS. 3A to 3C illustrate a torus surface. FIG. 3A is a diagram illustrating a torus surface. FIG. 3B is a diagram illustrating the locations of the small circle and the center axis. FIG. 3C is a diagram illustrating a circular arc used to form the PT surface.

As illustrated in FIG. 3A, the torus surface 70 is the surface of a solid 71 having a doughnut shape.

As illustrated in FIG. 3B, the torus surface 70 is the surface of a body of revolution generated by rotating a circle 72 around a straight line 73 as the axis that lies in the same plane as the circle 72 but does not intersect the circle 72. This circle 72 will be referred to as the small circle. As the circle is rotated, its circumference 74 moves to form the torus surface 70. The torus surface 70 is the locus of the circumference 74.

The PT surface is a surface partially cut out from the torus surface 70. While the torus surface 70 is formed by moving the circumference 74, the PT surface is formed by moving a part of the circumference 74.

FIG. 3C shows a circular arc 75. The circular arc 75 is a part of the circumference 74 of the circle 72. The PT surface is formed by rotating the circular arc 75 around the straight line 73.

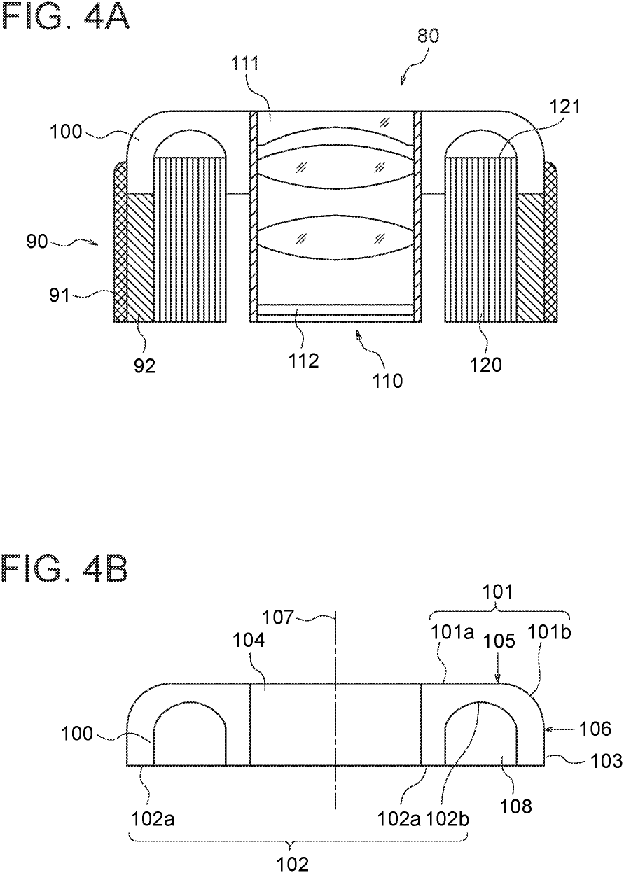

FIGS. 4A and 4B illustrate the second example of the basic structure. FIG. 4A is a cross sectional view of the end portion of the insert part. FIG. 4B is a cross sectional view of the end cover.

As illustrated in FIG. 4A, the end portion 90 of the insert part of the endoscope 80 is provided with an end cover 100. The end portion 90 of the insert part includes, for example, a sheathing tube 91 and a metal pipe 92. The sheathing tube 91 and the metal pipe 92 are in contact with the end cover 100.

In the end portion 90 of the insert part are disposed an imaging unit 100 and light guides 120. The imaging unit 110 includes an objective optical system 111 and an imager 112. Each light guide 120 has a light guide exit surface 121. The illumination light is emitted from the light guide exit surface 121.

The illumination light emitted from the light guide exit surface 121 enters the end cover 100. The end cover 100 is made of a transparent material. The transparent material used may be a resin. The illumination light incident on the end cover 100 is refracted and transmitted by the end cover 100 and then goes out from the end cover 100.

The endoscope 80 has two light guides 120 disposed at two locations that are symmetrical with respect to the imaging unit 110. Thus, the illumination light is cast in two directions toward the object.

As illustrated in FIG. 4B, the end cover 100 has a first surface 101, a second surface 102, and a side surface 103. The first surface 101 is the object side surface of the end cover 100. The second surface 102 is the operator side surface of the end cover 100. The side surface 103 is the outer circumferential surface of the end cover 100.

The end cover 100 has a through bore 104. An imaging unit 110 is inserted in and fixed to the through bore 104.

The first surface 101 includes a first flat surface 101a and a curved surface 101b. The curved surface 101b extends between the first flat surface 101a and the side surface 103.

The first flat surface 101a, the curved surface 101b, and the side surface 103 are arranged in such a way that they are smoothly connected to each other at their boundaries . The arrow 105 in FIG. 4B indicates the location of the boundary of the first surface 101a and the curved surface 101b. The arrow 106 in FIG. 4B indicates the location of the boundary of the curved surface 101b and the side surface 103.

FIG. 4B shows the center axis 107 of the end cover 100. The center of the through bore 104 is located on the center axis 47.

The second surface 102 of the end cover 100 includes non-illumination areas 102a and illumination areas 102b. The non-illumination area 102a is a flat surface.

The end cover 100 has recesses 108 located at positions opposed to the light guides 120. The illumination area 102b is constituted by the bottom surface of the recess 108. The illumination light emitted from the light guide exit surface 121 is incident on the illumination area 102b.

Since the end cover 100 is made of a transparent material, the illumination light incident on the illumination area 102b is refracted and transmitted through it and goes out from the first surface 101.

In the case of the second example of the basic structure, at least a portion of the illumination area is constituted by a PT surface. The entire surface of the illumination area 102b in the endoscope 80 is a PT surface.

Since the cross section of the torus surface is a small circle, the torus surface has a refractive power. Hence, the PT surface also has a refractive power. As the illumination area is constituted by, at least partly, by a PT surface, it can refract the illumination light emitted from the light guide exit surface. In consequence, it is possible to achieve a wide light distribution of illumination light.

As described above, the endoscope according to this embodiment has an imaging unit. The imaging unit includes an imager and an objective optical system. The imager has a rectangular image output area.

Since the image output area of the imager is rectangular in the image plane, the field of view is also essentially rectangular. In the following description, the diagonal direction in the image output area i.e., the area within which the subject image can be acquired from the imager, of the field of view, the image out put area is mentioned above will be referred to as the “diagonal direction of the field of view”, the direction of the long side of the image output area will be referred to as the “long side direction of the field of view”, and the direction of the short side of the image output area will be referred to as the “short side direction of the field of view”.

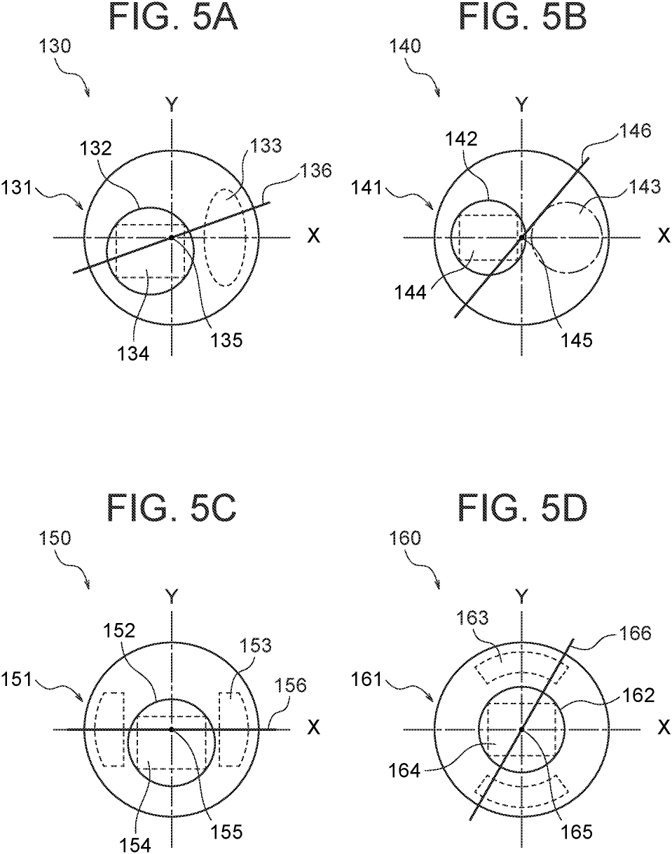

Now, the first specific cross section will be described with reference to FIGS. 5A, 5B, 5C, and 5D. FIGS. 5A 5B, 5C, and 5D are diagrams illustrating the end portion of the endoscope as seen from the object side.

FIG. 5A is a diagram illustrating a first example of the location of the first specific cross section. FIG. 5B is a diagram illustrating a second example of the location of the first specific cross section. FIG. 5C is a diagram illustrating a third example of the location of the first specific cross section. FIG. 5D is a diagram illustrating a fourth example of the location of the first specific cross section.

The first specific cross sections are a cross sections that contain the center axis of the end cover and intersect the light guide exit surface. The light guide exit surface is a surface with a limited expanse. Hence, there are a plurality of first specific cross sections.

The shape of the image output area is rectangular. The X axis in FIGS. 5A to 5D is set as the axis parallel to the long side of the rectangular image output area, and the Y axis is set as the axis parallel to the short side of the rectangular image output area.

As shown in FIG. 5A, when the end portion 131 of the insert part of the endoscope 130 is seen from the object side, an imaging unit 132 and a light guide exit surface 133 are disposed inside the end portion 131 of the insert part. The endoscope 130 includes one light guide.

The imaging unit 132 includes an objective optical system and an imager. The imager has an image output area 134. The center of the imaging unit 132 is offset from the center axis 135 of the end cover with respect to the X and Y directions.

The light guide exit surface 133 is disposed on the X-direction side of the imaging unit 132. The shape of the light guide exit surface 133 is elliptical.

The straight line 136 in FIG. 5A intersects the center axis 135 and the light guide exit surface 133. The cross section that contains the straight line 136 and is perpendicular to the plane of the drawing sheet of FIG. 5A is a cross section that contains the center axis 135 and intersects the light guide exit surface 133.

The first specific cross sections are cross sections that contain the center axis of the end cover and intersect the light guide exit surface 133. Therefore, the cross section that contains the straight line 136 and is perpendicular to the plane of the drawing sheet is a first specific cross section, and the straight line 136 represents the location of the first specific cross section in the end portion of the endoscope as seen from the object side.

As shown in FIG. 5B, when the end portion 141 of the insert part of the endoscope 140 is seen from the object side, an imaging unit 142 and a light guide exit surface 143 are disposed inside the end portion 141 of the insert part. The endoscope 140 includes one light guide.

The imaging unit 142 includes an objective optical system and an imager. The imager has an image output area 144. The center of the imaging unit 142 is offset from the center axis 145 of the end cover with respect to the X direction but not offset with respect to the Y direction.

The light guide exit surface 143 is disposed on the X-direction side of the imaging unit 142. The shape of the light guide exit surface 143 is circular.

The straight line 146 in FIG. 5B intersects the center axis 145 and is tangent to the light guide exit surface 143. In the present disclosure, this state shall also be included in the state defined by the statement that “the straight line 146 intersects the light guide exit surface 143”. The cross section that contains the straight line 146 and is perpendicular to the plane of the drawing sheet of FIG. 5B is a cross section that contains the center axis 145 and intersects the light guide exit surface 143.

The first specific cross sections are cross sections defined above. Therefore, the cross section that contains the straight line 146 and is perpendicular to the plane of the drawing sheet is a first specific cross section, and the straight line 146 represents the location of the first specific cross section in the end portion of the endoscope as seen from the object side.

As shown in FIG. 5C, when the end portion 151 of the insert part of the endoscope 150 is seen from the object side, an imaging unit 152 and light guide exit surfaces 153 of are disposed inside the end portion 151 of the insert part. The endoscope 150 includes two light guides.

The imaging unit 152 includes an objective optical system and an imager. The imager has an image output area 154. The center of the imaging unit 152 is not offset from the center axis 155 of the end cover with respect to the X direction but offset with respect to the Y direction.

The light guide exit surfaces 153 are disposed on two opposite sides of the imaging unit 152 with respect to the X direction. Each light guide exit surface 153 has the shape of a rectangle one side of which is replaced by an arc.

The straight line 156 in FIG. 5C intersects the center axis 155 and the light guide exit surfaces 153. The cross section that contains the straight line 156 and is perpendicular to the plane of the drawing sheet of FIG. 5C is a cross section that contains the center axis 155 and intersects the light guide exit surfaces 153.

The first specific cross sections are cross sections defined above. Therefore, the cross section that contains the straight line 156 and is perpendicular to the plane of the drawing sheet is a first specific cross section, and the straight line 156 represents the location of the first specific cross section in the end portion of the endoscope as seen from the object side.

As shown in FIG. 5D, when the end portion 161 of the insert part of the endoscope 160 is seen from the object side, an imaging unit 162 and light guide exit surfaces 163 are disposed inside the end portion 161 of the insert part. The endoscope 160 includes two light guides.

The imaging unit 162 includes an objective optical system and an imager. The imager has an image output area 164. The center of the imaging unit 162 is not offset from the center axis 165 with respect to either the X or Y direction.

The light guide exit surfaces 163 are disposed on two opposite sides of the imaging unit 162 with respect to the Y direction. Each light guide exit surface 163 has the shape of an annular sector.

The straight line 166 in FIG. 5D intersects the center axis 165 and the light guide exit surfaces 163. The cross section that contains the straight line 166 and is a plane that is perpendicular to the plane of the drawing sheet of FIG. 5D is a cross section that contains the center axis 165 and intersects the light guide exit surfaces 163.

The first specific cross sections are cross sections defined above. Therefore, the cross section that contains the straight line 166 and is perpendicular to the plane of the drawing sheet is a first specific cross section, and the straight line 166 represents the location of the first specific cross section in the end portion of the endoscope as seen from the object side.

The number and the shape of the light guide exit surfaces are not limited to those shown in FIGS. 5A to 5D. The direction and the amount of offset of the imaging unit from the center axis are not limited to those shown in FIGS. 5A to 5D. The relative positions of the light guide exit surfaces and the imaging unit is not limited to those shown in FIGS. 5A to 5D.

The endoscope according to the first embodiment has the basic structure described above and satisfies the following conditional expression (1) in at least one first specific cross section:

$$0 \leq \text{hA} / \text{r} \leq 0.5$$

where a partial torus surface is defined as a surface partially cut out from a torus surface, the torus surface is the surface of a body of revolution formed by rotating a circle in a plane about an axis of rotation that lies in the same plane and does not intersect the circle, this circle is referred to as a small circle, hA is the distance between a first intersection point and a second intersection point, the first intersection point is the point of intersection of the straight line passing through the center of the small circle and parallel to the center axis and the light guide exit surface, the second intersection point is the point of intersection of a ray passing through the boundary of the first flat surface and the curved surface and parallel to the center axis in the space between the light guide exit surface and the illumination area and the light guide exit surface, in the case where the second intersection point is located between the first intersection point and the side surface, the distance is represented by a value with a plus sign (namely, a positive value), and r is the radius of the small circle.

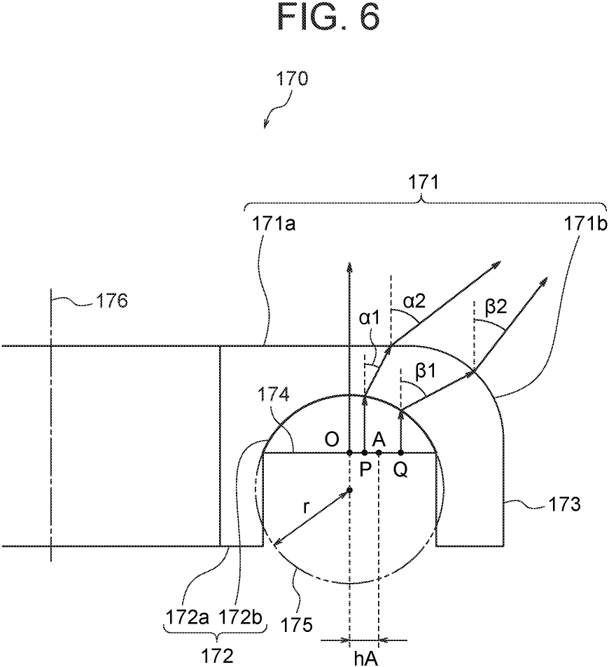

The parameters used in conditional expression (1) will be described below with reference to FIG. 6. FIG. 6 is a cross sectional view of an end cover.

The end cover 170 has a first surface 171, a second surface 172, and a side surface 173. The first surface 171 includes a first flat surface 171a and a curved surface 171b. The curved surface 171b extends between the first flat surface 171a and the side surface 173. The second surface 172 includes a non-illumination area 172a and an illumination area 172b. The illumination area 172b is opposed to a light guide exit surface 174.

The illumination area 172b of the end cover 170 is constituted entirely of a PT surface. In the cross section shown in FIG. 6, the illumination area 172b is constituted by a portion of a circle 175 having a radius r. This circle 175 is the small circle of the torus surface.

Now, the distance hA will be discussed.

In FIG. 6, the first intersection point is indicated as point O. The first intersection point is the point of intersection of the light guide exit surface 174 and the straight line passing through the center of the circle 175 and parallel to the center axis 176 of the end cover.

In FIG. 6, the second intersection point is indicated as point A. The ray passing through point A and parallel to the center axis 176 of the end cover is refracted by the illumination area 172b and passes through the boundary of the first flat surface and the curved surface.

The parameter hA is the distance between the first intersection point and the second intersection point. In the case where the second intersection point is located between the first intersection point and the side surface, this distance is represented by a value with a plus sign (namely, a positive value). In the case where the second intersection point is located between the first intersection point and the center axis, this distance is represented by a value with a minus sign (namely, a negative value). In the case shown in FIG. 6, point A is located between point O and the side surface 173. Therefore, the sign of the value of hA is plus.

Rays are emitted from the light guide at various angles. The rays emitted from the light guide exit surface 174 are refracted by the illumination area 172b to increase the angle of distribution of light.

The rays going out from the illumination area 172b enter the first flat surface 171a and the curved surface 171b of the first surface 171. Since the first flat surface 171a is flat, it refracts rays in such directions as to further increase the angle of distribution of light. Since the curved surface 171b is a convex surface and has a positive refractive power, it refracts rays in such directions as to decrease the angle of distribution of light.

For the sake of simplicity of description, the rays emitted from the light guide exit surface in the direction perpendicular to the light guide exit surface will be described with reference to FIG. 6. Rays are emitted from the light guide exit surface in various directions. Among the rays of the illumination light emitted from the light guide, the rays emitted in the direction perpendicular to the light guide exit surface have the highest light intensity.

Point O is the point of intersection of the light guide exit surface 174 and the straight line passing through the center of the circle 175 and parallel to the center axis 176 of the end cover. Points P and Q are points on the light guide exit surface 174 and closer to the outer circumference of the endoscope than point O. They are located in the order of point P and then point Q along the direction from point O to the outer circumference.

The ray emitted from point O in the direction parallel to the center axis 176 is not refracted by either the illumination area 172b or the first flat surface 171a but travels in a straight line.

The ray emitted from point P in the direction parallel to the center axis 176 is refracted by the illumination area 172b. The angle formed by the center axis 176 and the refracted ray is represented by α1. Thereafter, this ray is further refracted by the first flat surface 171a. The angle formed by the center axis 175 and the refracted ray is represented by α2.

The ray emitted from point Q in the direction parallel to the center axis 176 is refracted by the illumination area 172b. The angle formed by the center axis 176 and the refracted ray is represented by β1. Thereafter, this ray is further refracted by the curved surface 171b. The angle formed by the center axis 176 and the refracted ray is represented by β2.

If β2 < α2 as shown in FIG. 6, the light intensity becomes high at specific angles in the angular distribution of the illumination light intensity. This results in uneven illumination. If the angular difference between the illumination light going out from the first flat surface 171a and the illumination light going out from the curved surface 171b is large, unevenness of illumination tends to be noticeable.

Light is emitted from the light guide actually at various angles within the angle range represented by the numerical aperture. Point A is the location of the ray on the light guide exit surface 174 that is emitted from the light guide exit surface 174 in the direction parallel to the center axis 176 of the end cover and passes through the boundary of the first flat surface 171a and the curved surface 171b, and hA is the distance between point O and point A. If the distance hA falls within the range defined by conditional expression (1), unevenness of illumination is not so noticeable as to cause a problem in observation.

If the value of hA becomes larger, the entirety of the illumination light emitted from the light guide passes through the first flat surface 171a. Then, no illumination light is emitted from the curved surface. This will eliminate unevenness of illumination but lead to an increased outer diameter of the end portion of the endoscope, which is disadvantageous for the endoscope.

Now, technical meaning of conditional expression (1) will be described. In the following description, the range up to the maximum unevenness of illumination is discussed. In the following description, the term “ease of insertion” will be used to refer to the ease of insertion of the insert part into the body.

If the value of hA/r exceeds the upper bound of conditional expression (1), the second intersection point is too far from the center axis, or the diameter of the small circle is too small.

If the second intersection point is far from the center axis, the difference between the angle of the illumination light going out from the first flat surface 171a and the angle of the illumination light going out from the curved surface is large. In consequence, unevenness of illumination tends to be noticeable. Then, it is difficult to achieve satisfactory observation to cause a problem in making accurate diagnosis.

Moreover, if the second intersection point is too far from the center axis, the curved surface is too far from the center axis. In consequence, the outer diameter of the insert part becomes large to deteriorate the ease of insertion.

If the radius of the small circle is too small, the curvature radius of the PT surface is small, and the refractive power of the illumination area is high. Then, the distribution of the illumination light going out from the illumination area will be broad.

However, in the case where the illumination area is entirely constituted by a PT surface, the extent of the illumination area is small. In the case where the extent of the illumination area is small, the extent of the exit surface must be made small. This will result in an insufficient amount of illumination light.

If the extent of the exit surface is not made small, the illumination area is constituted by, for example, a PT surface and a flat surface disposed outside the PT surface.

Then, the illumination light emitted from the light guide exit surface enters the PT surface and the flat surface. The flat surface has no refractive power. Therefore, the degree of divergence of the illumination light going out from the fat surface is smaller than that in the case where PT surface is used.

A portion of the illumination light passing through the flat surface is incident on the curved surface. The illumination light incident on the curved surface is refracted toward the center axis. In consequence, degree of unevenness of illumination is increased.

If the value of hA/r falls below the lower bound of conditional expression (1), the second intersection point is too close to the center axis, or the radius of the small circle is too large.

If the second intersection point is too close to the center axis, even if the light distribution angle is large in the illumination area, most of the rays are incident on the curved surface and refracted in such directions as to narrow the distribution angle. In consequence, the distribution of the illumination light will be narrow.

If the radius of the small circle is too large, the curvature radius of the PT surface is too large. Then, the refractive power of the illumination area is low, resulting in a narrow distribution of illumination light.

If the distribution of illumination light is narrow, the amount of illumination light will be insufficient in the peripheral area of the field of view. Then, it will be difficult to achieve satisfactory observation in the peripheral area of the field of view to cause a problem in making accurate diagnosis.

An endoscope according to a second embodiment has the basic structure described above and satisfies the following conditional expression (2) in at least one first specific cross section:

$$0 \leq \left(\text{hA} \times {\text{n}}^{2}\right) / \text{r} \leq 1.36$$

where a partial torus surface is defined as a surface partially cut out from a torus surface, the torus surface is the surface of a body of revolution formed by rotating a circle in a plane about an axis of rotation that lies in the same plane and does not intersect the circle, this circle is referred to as a small circle, hA is the distance between a first intersection point and a second intersection point, the first intersection point is the point of intersection of the straight line passing through the center of the small circle and parallel to the center axis and the light guide exit surface, the second intersection point is the point of intersection of a ray passing through the boundary of the first flat surface and the curved surface and parallel to the center axis in the space between the light guide exit surface and the illumination area and the light guide exit surface, in the case where the second intersection point is located between the first intersection point and the side surface, the distance is represented by a value with a plus sign (namely, a positive value), n is the refractive index of the material of the end cover for the e-line, and r is the radius of the small circle.

The technical meaning of conditional expression (2) is the same as that of conditional expression (2).

The endoscopes according to the first and second embodiments can achieve a wide distribution of illumination light. In consequence, the illumination light is cast sufficiently along not only the long side direction of the field of view but also the diagonal direction of the field of view.

The imaging unit of the endoscope according to this embodiment has an imager. The imager has a rectangular image output area. A first cross section is a cross section expressed by the following equation (3), and a second cross section is a cross section that satisfies the following conditional expression (4). The first and second cross sections are first specific cross sections, and conditional expression (1) is satisfied in the first and second cross sections.

$$\text{φ1=0}$$

$$0.2 \leq \text{φ2} / \text{ε} \leq 0.7$$

where φ1 is the angle formed by a second specific cross section and the first cross section, φ2 is the angle formed by the second specific cross section and the second cross section, the second specific cross section is the cross section parallel to the long side of the image output area and containing the center axis, ε is the angle formed by a cross section containing the long side of the image output area and a cross section containing the diagonal of the image output area.

The imaging unit of the endoscope according to this embodiment has an imager. The imager has a rectangular image output area.

The first cross section is a cross section expressed by equation (3), and the second cross section is a cross section that satisfies conditional expression (4).

Equation (3) is an equation relating to the angle formed by the second specific cross section and the first cross section. Conditional expression (4) is a conditional expression relating to the angle formed by the second specific cross section and the second cross section. The second specific cross section is the cross section parallel to the long side of the image output area and containing the center axis.

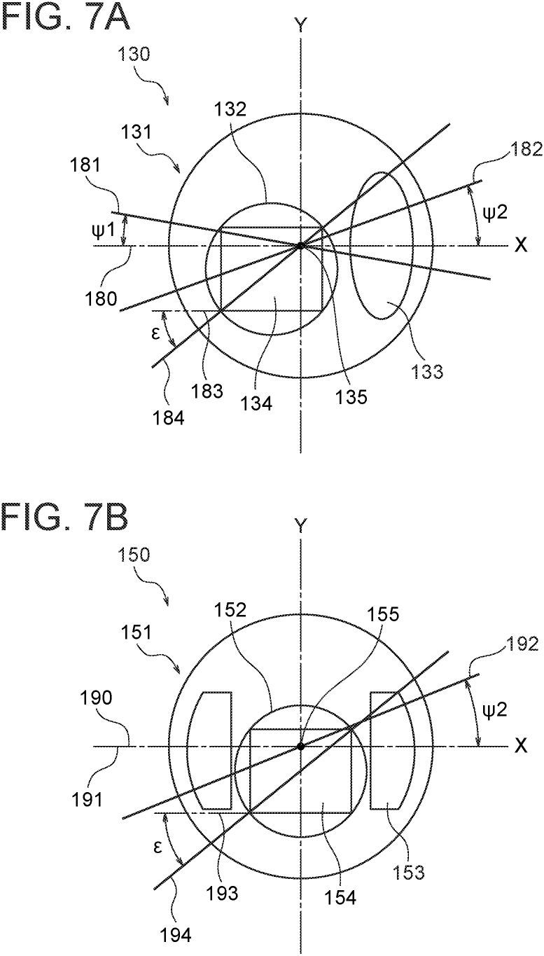

The parameters used in equation (3) and conditional expression (4) will be described with reference to FIGS. 7A and 7B. FIGS. 7A and 7B are diagrams illustrating the end portion of the endoscope as seen from the object side.

FIG. 7A is a diagram showing a first example of the locations of the first and second specific cross sections. The elements same as those in FIG. 5A are denoted by the same reference numeral and will not be described further. FIG. 7B is a diagram showing a second example of the locations of the first and second specific cross sections. The elements same as those in FIG. 5B are denoted by the same reference numeral and will not be described further.

As shown in FIG. 7A, an imaging unit 132 and a light guide exit surface 133 are disposed in the end portion 131 of the insert part of the endoscope 130. The imaging unit 132 has an imager. The imager has an image output area 134. The shape of the image output area 134 is rectangular.

The line 180 in FIG. 7A is the straight line parallel to the long side of the image output area 134 and intersecting the center axis 135. The second specific cross section is the cross section that is parallel to the long side of the image output area and contains the center axis of the end cover. Therefore, the cross section containing the straight line 180 and perpendicular to the plane of the drawing sheet of FIG. 7A is the second specific cross section.

Referring to the straight line 181 in FIG. 7A, the cross section containing the straight line 181 and perpendicular to the plane of the drawing sheet of FIG. 7A contains the center axis 135 and intersects the light guide exit surface 133. Therefore, the cross section containing the straight line 181 and perpendicular to the plane of the drawing sheet may be regarded as a first specific cross section. If this cross section is the first cross section, the angle φ1 formed by the straight line 180 and the straight line 181 is the angle formed by the second specific cross section and the first cross section.

Since the first cross section and the second specific cross section intersect, the angle φ1 they form is not equal to 0°.

As above, the second specific cross section is the cross section containing the straight line 180 and perpendicular to the plane of the drawing sheet. Referring to the straight line 182 in FIG. 7A, the cross section containing the straight line 182 and perpendicular to the plane of the drawing sheet of FIG. 7A contains the center axis 135 and intersects the light guide exit surface 133. Therefore, the cross section containing the straight line 182 and perpendicular to the plane of the drawing sheet may be regarded as a first specific cross section. If this cross section is the second cross section, the angle φ2 formed by the straight line 180 and the straight line 182 is the angle formed by the second specific cross section and the second cross section.

The cross section containing the straight line 183 in FIG. 7A and perpendicular to the plane of the drawing sheet is parallel to the long side of the image output area 134. The cross section containing the straight line 184 in FIG. 7A and perpendicular to the plane of the drawing sheet contains the diagonal of the image output area 134. Therefore, the angle ε formed by the straight line 183 and the straight line 184 is the angle formed by the cross section containing the long side of the image output area and the cross section containing the diagonal of the image output area.

As shown in FIG. 7A, the angle φ2 is a little greater than half of the angle ε.

Referring to the example shown in FIG. 7B, an imaging unit 152 and light guide exit surfaces 153 are disposed in the end portion 151 of the insert part of the endoscope 150. The imaging unit 152 has an imager. The imager has an image output area 154. The shape of the image output area 154 is rectangular.

The straight line 190 in FIG. 7B is the straight line parallel to the long side of the image output area 154 and intersecting the center axis 155. The second specific cross section is the cross section that is parallel to the long side of the image output area and contains the center axis of the end cover. Therefore, the cross section containing the straight line 190 and perpendicular to the plane of the drawing sheet of FIG. 7B is the second specific cross section.

Referring to the straight line 191 in FIG. 7B, the cross section containing the straight line 191 and perpendicular to the plane of the drawing sheet of FIG. 7B contains the center axis 155 and intersects the light guide exit surface 153. Therefore, the cross section containing the straight line 191 and perpendicular to the plane of the drawing sheet may be regarded as a first specific cross section. If this cross section is the first cross section, the angle φ1 formed by the straight line 190 and the straight line 191 is the angle formed by the second specific cross section and the first cross section.

Since the first cross section and the second specific cross section are the same plane, the angle φ1 formed by them is 0°.

As above, the second specific cross section is the cross section containing the straight line 190 and perpendicular to the plane of the drawing sheet. Referring to the straight line 192 in FIG. 7B, the cross section containing the straight line 192 and perpendicular to the plane of the drawing sheet of FIG. 7B contains the center axis 155 and intersects the light guide exit surface 153. Therefore, the cross section containing the straight line 192 and perpendicular to the plane of the drawing sheet may be regarded as a first specific cross section. If this cross section is the second cross section, the angle φ2 formed by the straight line 190 and the straight line 192 is the angle formed by the second specific cross section and the second cross section.

The cross section containing the straight line 193 in FIG. 7B and perpendicular to the plane of the drawing sheet is parallel to the long side of the image output area 154. The cross section containing the straight line 194 in FIG. 7B and perpendicular to the plane of the drawing sheet contains the diagonal of the image output area 154. Therefore, the angle ε formed by the straight line 193 and the straight line 194 is the angle formed by the cross section containing the long side of the image output area 154 and the cross section containing the diagonal of the image output area 154.

As shown in FIG. 7B, the angle φ2 is a little greater than half of the angle ε.

The first cross section is the cross section expressed by equation (3). The cross section expressed by equation (3) is the cross section that is parallel to the long side of the image output area and contains the center axis. The first cross section extends along the long side direction of the field of view.

The first cross section is a first specific cross section, and conditional expression (1) is satisfied in the first cross section. Therefore, it is possible to achieve a wide distribution of illumination light along the long side direction of the field of view while keeping unevenness of illumination small.

The second cross section is a cross section that satisfies conditional expression (4). The cross section that satisfies conditional expression (4) is a cross section that intersects the long side of the image output area and contains the center axis. This cross section is oriented in a direction crossing the long side direction of the field of view. The direction crossing the long side direction of the field of view is a direction close to the diagonal direction of the field of view.

The second cross section is a first specific cross section, and conditional expression (1) is satisfied in the second cross section. Therefore, it is possible to achieve a wide distribution of illumination light along the direction close to the diagonal direction of the field of view while keeping unevenness of illumination small.

As above, if equation (3) and conditional expression (4) are satisfied, unevenness of illumination light can be kept small along almost all the directions.

If the value of φ2 / ε exceeds the upper bound of conditional expression (4), the angle formed by the second cross section and the second specific cross section is so large that the second cross section comes close to or even exceeds the diagonal direction. Alternatively, if the value of φ2 / ε exceeds the upper bound of conditional expression (4), the length of the short side of the image output area is too short.

The second cross section is a first specific cross section. The first specific cross section is a cross section that contains the center axis of the end cover and intersects the light guide exit surface. Therefore, it is presumed that the second cross section intersects the light guide exit surface.

When the angle formed by the second cross section and the second specific cross section is large, the light guide exit surface extends largely along the short side direction of the image output area, and a large amount of illumination light illuminates the area outside the field of view. If the amount of illumination light emitted from the light guide exit surface that illuminates the area outside the field of view increases, the efficiency of use of the illumination system (which will be referred to as “illumination efficiency” hereinafter) decreases, and the illumination light will dim even if the same number of optical fibers are used in the light guide.

Moreover, the number of optical fibers that do not contribute to illumination will increase. The number of optical fibers used affects the outer diameter of the end portion of the insert part and the flexibility thereof. If the number of optical fibers that do not contribute to illumination is large, the outer diameter of the end portion of the insert part will become large uselessly. Moreover, the flexibility of the end portion of the insert part will decrease, leading to increased risk of breakage of optical fibers.

If the short side of the image output area is too short, the imaging area will be too narrow along the short side direction of the field of view.

If the value of φ2 / ε falls below the lower bound of conditional expression (4), the second cross section is too close to the second specific cross section, or the length of the short side of the image output area is too long.

The first cross section coincides with the second specific cross section. If the second close section is too close to the second specific cross section, the difference between the second cross section and the first cross section is too small. Then, it is possible to achieve a wide distribution of illumination light while keeping unevenness of illumination small along the long side direction of the field of view. However, it is difficult to achieve a wide distribution of illumination light while keeping unevenness of illumination small along directions crossing the long side direction of the field of view.

If the length of the short side of the image output area is too long, the imager is necessitated to be large, resulting in an increased outer diameter of the insert part. Then, the ease of insertion will be deteriorated.

The endoscope according to this embodiment satisfies the following conditional expression (5):

$$0 < \left(\text{dy} \times \text{n}\right) / \text{R} < 0.5$$

where dy is the distance between the first intersection point and a third intersection point, the third intersection point is the point of intersection of the straight line passing through the center of curvature of the curved surface in the first surface and parallel to the center axis and the light guide exit surface, in the case where the third intersection point is located between the first intersection point and the side surface, the distance is represented by a value with a plus sign (namely, a positive value), n is the refractive index of the material of the end cover for the e-line, and R is the curvature radius of the curved surface in the first surface.

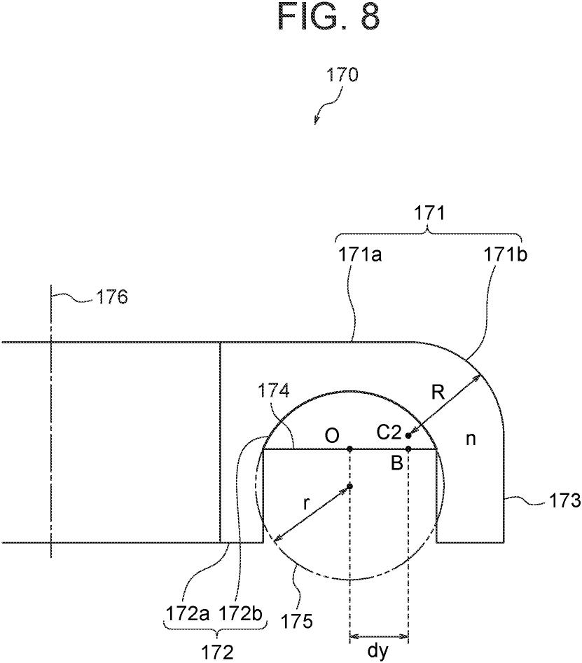

The parameters used in conditional expression (5) will be described below with reference to FIG. 8. FIG. 8 is a cross sectional view of an end cover. The components in FIG. 8 that are the same as those in FIG. 6 are denoted by the same reference numerals and will not be described further.

In FIG. 8, n is the refractive index of the material of the end cover 170 for the e-line, and R is the curvature radius of the curved surface 171b.

In FIG. 8, the third intersection point is indicated as point B. The third intersection point is the point of intersection of the straight line passing through the center of curvature C2 of the curved surface 171b and parallel to the center axis 176 and the light guide exit surface 174.

In FIG. 8, dy is the distance between the first intersection point and the third intersection point. In the case where the third intersection point is located between the first intersection point and the side surface, the distance is represented by a value with a plus sign (namely, a positive value). In the case shown in FIG. 8, intersection point B is located between intersection point O and the side surface 173. Therefore, the sign of the value of dy is plus.

If the value of (dy × n)/R exceeds the upper bound of conditional expression (5), the third intersection point is too far from the center axis, or the curvature radius of the curved surface is too small.

If the third intersection point is too far from the center axis, unduly large unevenness of illumination will result, as described above in the description of the technical meaning of conditional expression (1). Moreover, the ease of insertion will be deteriorated.

If the curvature radius of the curved surface is too small, the angular difference between the illumination light going out from the first flat surface 171a and the illumination light going out from the curved surface 171b is so large that unevenness of illumination tends to be noticeable. Then, it will be difficult to achieve satisfactory observation to cause a problem in making accurate diagnosis.

If the curvature radius of the curved surface is too small, the end portion of the insert part has a square-cornered shape, deteriorating the ease of insertion.

If the value of (dy × n)/R falls below the lower bound of conditional expression (5), the third intersection point is too close to the center axis, or the curvature radius of the curved surface is infinity.

If the third intersection point is too close to the center axis, the distribution of illumination light will be narrow, as described in the above description of the technical meaning of conditional expression (1).

If the curvature radius of the curved surface is infinity, it is impossible to design the endoscope.

The endoscope according to this embodiment satisfies the following conditional expression (6):

$$- 0.15 < \text{n} \times \left(\text{R} - \text{r} - \text{t}\right) < 0$$

where n is the refractive index of the material of the end cover for the e-line, R is the curvature radius of the curved surface in the first surface, r is the radius of the small circle, and t is the shortest distance between the first flat surface and the illumination area.

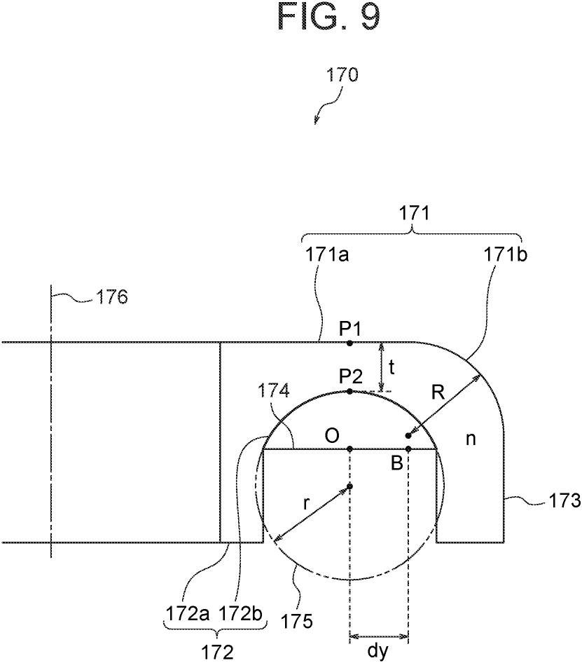

The parameters used in conditional expression (6) will be described with reference to FIG. 9. FIG. 9 is a cross sectional view of an end cover. The components in FIG. 9 that are the same as those in FIG. 8 are denoted by the same reference numerals and will not be described further.

In FIG. 9, the distance t is shown as the distance between point P1 and point P2. The distance between point P1 and point P2 is the shortest distance between the first flat surface 171a and the illumination area 172b. The sign of the value t is always plus.

If the value of n × (R - r - t) exceeds the upper bound of conditional expression (6), the curvature radius of the curved surface is too large, or the radius of the small circle is too small.

If the curvature radius of the curved surface is too large, the curved surface is too far from the center axis. Then, the outer diameter of the insert part is too large, deteriorating the ease of insertion.

If the radius of the small circle is too small, the amount of illumination light will be insufficient, as described in the above description of the technical meaning of conditional expression (1).

If the value of n × (R - r - t) falls below the upper bound of conditional expression (6), the curvature radius of the curved surface is too small, or the radius of the small circle is too large.

If the curvature radius of the curved surface is too small, it will be difficult to make the unevenness of illumination small, or the ease of insertion will be deteriorated, as described in the description of the technical meaning of conditional expression (5).

If the radius of the small circle is too large, the distribution of illumination light will be narrow, as described in the above description of the technical meaning of conditional expression (1).

The illumination area of the endoscope according to this embodiment has a second flat surface and a partial torus surface, and the second flat surface is located closer to the center axis than the partial torus surface.

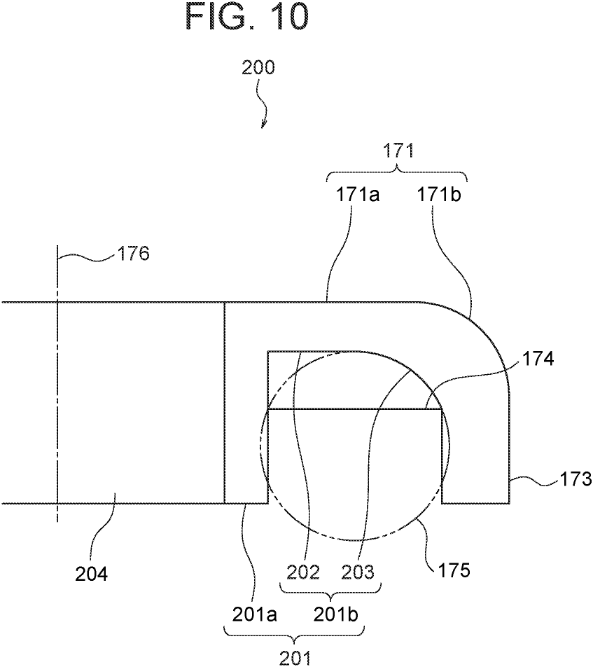

FIG. 10 is a cross sectional view of the end cover. The components in FIG. 10 that are the same as those in FIG. 6 are denoted by the same reference numerals and will not be described further.

The second surface 201 of the end cover 200 includes a non-illumination area 201a and an illumination area 201b. The illumination area 201b includes a second flat surface 202 and a PT surface 203. The second flat surface 202 is located closer to the center axis 176 than the PT surface 203

The illumination light emitted from the light guide exit surface 174 is incident on the illumination area 201b. Since the illumination light emitted from the light guide exit surface 174 is divergent light, which is incident on the second flat surface 202 and the PT surface 203.

The divergent light incident on the second flat surface 202 travels toward the first surface 171. The second flat surface 202 does not have a refractive power. Therefore, the degree of divergence of the light emitted from the second flat surface 202 is lower than that in the case where a PT surface is used.

The end cover 200 is provided with a through bore 204. As the degree of divergence of the illumination light travelling toward the first surface 171 is high, the amount of illumination light travelling toward the through bore 204 increases.

In the through bore 204 is provided an imaging unit. When the illumination light emitted from the second surface 201 reaches the through bore 204, absorption and reflection of light occur on the side surface of the through bore 204 and the side surface of the imaging unit.

Absorption of light generates heat, and the temperature of the end portion of the insert part rises consequently.

The light reflected as above is totally reflected by or transmitted through the first flat surface 171a. Whether reflected or transmitted, the illumination light travels toward the area outside the field of view or the area inside the field of view.

In the case where the illumination light travels toward the area outside the field of view, the amount of illumination light that falls outside the field of view is large. Then, the illumination efficiency is low. In the case where the illumination light travels toward the area inside the field of view, unevenness of illumination will result.

As described above, the second flat surface 202 does not have a refractive power. In consequence, in the case of this end cover 200, the amount of illumination light that travels toward the through bore 204 is small. Therefore, it is possible to prevent or reduce the generation of heat, deterioration in the illumination efficiency, and unevenness of illumination.

The divergent light incident on the PT surface 203 travels toward the first surface 171. The PT surface 203 functions as a surface having a negative refractive power. Hence, the divergent light incident on the PT surface 203 diverges further. The divergent light having passed through the PT surface 203 enters the first flat surface 171a and the curved surface 171b.

The first flat surface 171a does not have a refractive power. Therefore, the light going out from the first flat surface 171a is divergent light. The illumination light emitted from the first flat surface 171a is divergent light. Therefore, a wide distribution of illumination light can be achieved.

The imaging unit of the endoscope according to this embodiment has an imager. The imager has a rectangular image output area. The outer circumference of the light guide exit surface is composed of an inner edge, an intermediate edge, and an outer edge. The inner edge is located closer to the center axis than the outer edge, and the intermediate edge is located between the inner edge and the outer edge. The outer edge has the shape of an arc of a circle having its center on the center axis. The endoscope satisfies the following conditional expression (7):

$$0.7 < \text{θ} / \text{ε} < 1.2$$

where θ is the angle formed by a second specific cross section and a third cross section, the second specific cross section is the cross section containing the center axis and parallel to the long side of the image output area, the third cross section is the cross section containing the point of intersection of the outer edge and the intermediate edge and the center axis, ε is the angle formed by a cross section containing the long side of the image output area and a cross section containing the diagonal of the image output area.

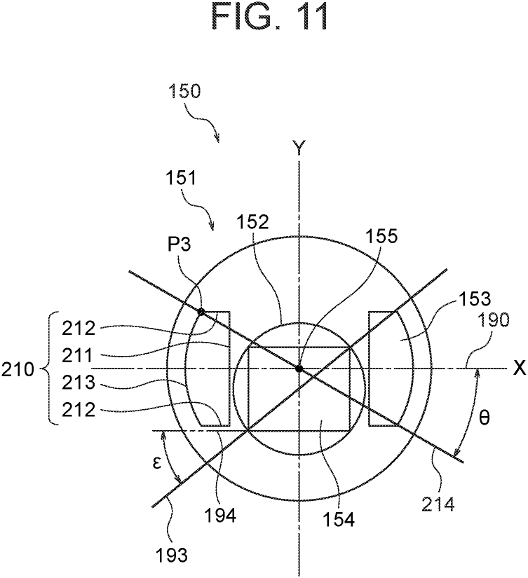

FIG. 11 is a diagram illustrating an end portion of the endoscope as seen from the object side. The components in FIG. 11 that are the same as those in FIG. 7B are denoted by the same reference numerals and will not be described further.

The shape of a light guide exit surface 153 in FIG. 11 is a rectangle one side of which is replaced by an arc. The outer circumference 210 of the light guide exit surface 153 is composed of an inner edge 211, intermediate edges 212, and an outer edge 213.

The inner edge 211 is located closer to the center axis 155 than the outer edge 213. The intermediate edges 212 are located between the inner edge 211 and the outer edge 213. The inner edge 211 and the intermediate edges 212 are straight. The outer edge 213 has a shape of an arc of a circle having its center located on the center axis 155.

Replacement of one side of the rectangular shape of the light guide exit surface 153 by an arc makes the area of the light guide exit surface 153 larger than the rectangular shape, and it is possible to deliver the illumination light emitted from the light guide exit surface 153 to the peripheral region of the illumination area. Thus, it is possible to diverge the illumination light emitted from the light guide exit surface 153 to widen the distribution of illumination light. In consequence, excellent observation is ensured in the peripheral area of the field of view.

The arc shape of the outer edge 213 improves the ease of machining of the light guide exit surface 153. The improvement in the ease of machining of the light guide exit surface 153 enables highly precise machining of the light guide exit surface 153. This results in a reduction in the dimensional error of the light guide exit surface 153.

It is also possible to process the shape of the recess of the end cover with high precision. Since the dimensional error of the end cover can be reduced, the end cover and the light guide exit surface 153 can be assembled easily.

In the arrangement shown in FIG. 11, two light guide exit surfaces 153 are disposed on two sides of the imaging unit 152. However, the endoscope may include only one light guide exit surface 153 provided on one side of the imaging unit 152.

The parameters used in conditional expression (7) will be described below with reference to FIG. 11.

As above, ε is the angle formed by a cross section containing the long side of the image output area and a cross section containing the diagonal of the image output area. The cross section containing the straight line 190 in FIG. 11 and perpendicular to the plane of the drawing sheet of FIG. 11 is the second specific cross section.

Referring to the straight line 214 in FIG. 11, the cross section containing the straight line 214 and perpendicular to the plane of the drawing sheet of FIG. 11 contains the point of intersection P3 of the outer edge 213 and one of the intermediate edges 212 and the center axis 155. The third cross section is the cross section containing the point of intersection of the outer edge and the intermediate edge and the center axis. Therefore, the cross section containing the straight line 214 and perpendicular to the plane of the drawing sheet may be regarded as the third cross section. Hence, the angle θ formed by the straight line 190 and the straight line 214 is the angle formed by the second specific cross section and the third cross section.

The intersection point P3 defines the end of the light guide exit surface 153 with respect to the short side direction of the image output area 154. As the intersection point P3 shifts in the upward direction away from the X axis, the light guide exit surface 153 expands along the short side direction of the image output area 154.

If conditional expression (7) is satisfied, it is possible to achieve a wide distribution of illumination light. Then, illumination light is delivered sufficiently not only along the long side direction of the field of view but also the short side direction of the field of view. The amount of illumination light cast to the area outside the field of view with respect to the short side direction is small. Thus, the illumination efficiency is improved.

If the value of θ / ε exceeds the upper bound of conditional expression (7), the light guide exit surface is too large along the short side direction of the image output area, or the length of the short side of the image output area is too short. If the light guide exit surface is too large along the short side direction of the image output area, the illumination efficiency is decreased, as described in the description of the technical meaning of conditional expression (4) . If the area of the light guide exit surface increases, the number of optical fibers increases, necessitating a large outer diameter of the endoscope. If the number of optical fibers is increased without increasing the outer diameter of the endoscope, the possibility of breakage of optical fibers at a curved portion of the insert part of the endoscope will increase.

If the length of the short side of the image output area is too short, the imaging range along the short side direction of the field of view is too short.

If the value of θ / ε falls below the lower bound of conditional expression (7), the light guide exit surface is too small along the short side direction of the image output area, or the length of the short side of the image output area is too long. This will result in a narrow distribution of illumination light along the diagonal direction of the field of view. Moreover, the amount of illumination light will be insufficient.

In consequence, it will be difficult to achieve satisfactory observation particularly in the diagonally peripheral area of the field of view to cause a problem in making accurate diagnosis.

If the length of the short side of the image output area is too long, the ease of insertion will be deteriorated, as described in the description of the technical meaning of conditional expression (3).

The outer circumference of the light guide exit surface in the endoscope according to this embodiment is composed of an inner edge, an intermediate edge, and an outer edge. The inner edge is located closer to the center axis than the outer edge, and the intermediate edge is located between the inner edge and the outer edge. The outer edge has the shape an arc of a circle having its center on the center axis. The endoscope satisfies the following conditional expression (8) in at least one first specific cross section:

$$0.6 < \text{L} / \text{r} \leq 1.0$$

where L is the distance between the first intersection point and the outer edge, and r is the diameter of the small circle.

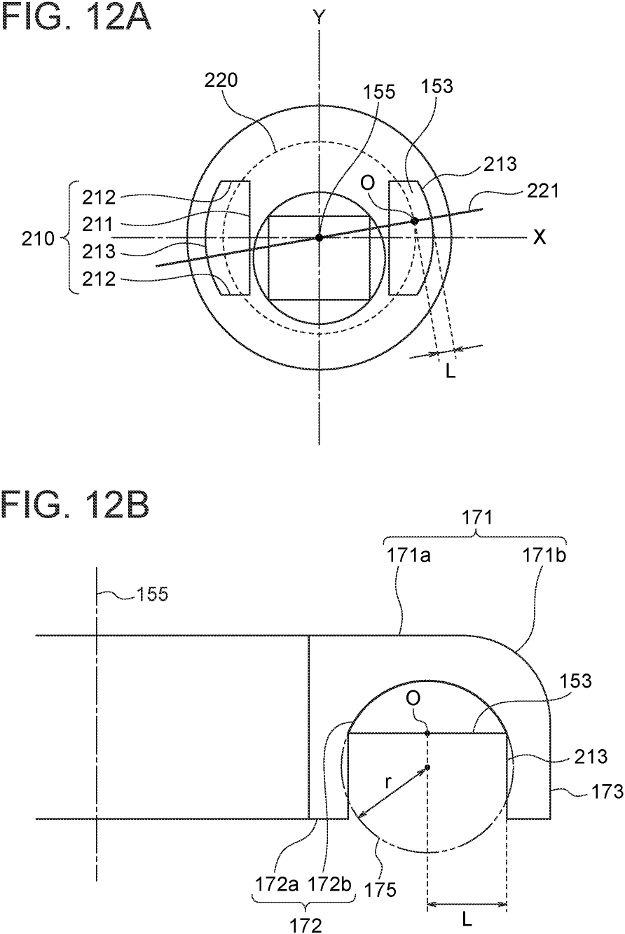

FIGS. 12A and 12B are diagrams illustrating the end portion of an insert part and an end cover . FIG. 12A is a diagram showing the end portion of the endoscope as seen from the object side. FIG. 12B is a cross sectional view of the end cover. The components in FIGS. 12A and 12B that are the same as those in FIG. 6 are denoted by the same reference numerals and will not be described further.

The circle 220 drawn by a broken line in FIG. 12A is the circle formed as the locus of the center of the circle 175 shown in FIG. 12B when the circle 175 is rotated about the center axis 155.

The straight line 221 in FIG. 12A is the line passing through the center axis 155 and intersecting the light guide exit surface 153. The cross section containing the straight line 221 and perpendicular to the plane of the drawing sheet of FIG. 12A is a cross section containing the center axis 155 and intersecting the light guide exit surface 153.

The first specific cross section is a cross section that contains the center axis of the end cover and intersects the light guide exit surface. Therefore, the cross section containing the straight line 221 and perpendicular to the plane of the drawing sheet of FIG. 12A is a first specific cross section, and the straight line 221 indicates the location of this first specific cross section in the end portion of the endoscope as seen from the object side.

The parameters used in conditional expression (8) will be described below.

In FIGS. 12A and 12B, intersection point O is the first intersection as described above, and L is the distance between the first intersection point and the outer edge 213.

If the value of L/r exceeds the upper bound of conditional expression (8), the distance between the first intersection point and the outer edge is too long, or the radius of the small circle is too small.

If the distance between the first intersection point and the outer edge is too long, the illumination area cannot be composed only of a PT surface. For example, the illumination area may be composed of a PT surface and a flat surface provided on the outer side of the PT surface. Then, large unevenness of illumination will result, as described in the description of the technical meaning of conditional expression (1).

In consequence, it will be difficult to achieve satisfactory observation in the peripheral area of the field of view to cause a problem in making accurate diagnosis.

If the light guide exit surfaces are arranged along the long side direction of the image output area, the light guide exit surfaces extend too largely along the long side direction of the image output area. This will make the outer diameter of the insert part too large to deteriorate the ease of insertion.

If the diameter of the small circle is too small, the amount of illumination light is insufficient, as described in the description of the technical meaning of conditional expression (1).

If the value of L/r falls below the lower bound of conditional expression (8), the distance between the first intersection point and the outer edge is too short, or the radius of the small circle is too large.

If the distance between the first intersection point and the outer edge is too short, the expanse of the light guide exit surface is too narrow in the first specific cross section. Then the amount of illumination light will be insufficient, or the distribution of illumination light will be narrow.

In consequence, it will be difficult to achieve satisfactory observation in the peripheral area of the field of view to cause a problem in making accurate diagnosis.

If the radius of the small circle is too large, the distribution of illumination light will be narrow, as described in the description of the technical meaning of conditional expression (1).

The endoscope according to this embodiment satisfies the following conditional expression (9):

$$10 < \text{a} / \text{r} < 16$$

where a is the outer diameter of the end cover, and r is the radius of the small circle.

The outer diameter of the insert part of the endoscope may vary depending on the purpose of using the endoscope, even if the object to be observed is the same. For example, otorhinology endoscopes include endoscopes designed only for the purpose of observation and endoscopes designed for observation and treatment.

Endoscopes designed only for the purpose of observation are not provided with a channel for treatment tools . For this reason, they can use an imager having a large image output area.

Imagers having a large image output area can have a large number of pixels. Therefore, they can capture high-quality images. However, when an imager having a large image output area is used, the outer diameter of the insert part tends to be large.

Endoscopes for gastric observation includes nasal intubation endoscopes and oral intubation endoscopes. In the case of the oral intubation endoscopes, the insert part is inserted through the mouth. In the case of the nasal intubation endoscopes, the insert part is inserted through the nose.

Between the nose and the mouth, the space through which the insert part is passed is narrow. Therefore, the outer diameter of nasal intubation endoscopes is generally smaller than the outer diameter of oral intubation endoscopes. If the outer diameter of the insert part is small, it is not possible to use an imager having a large image output area.

For this reason, the number of pixels of imagers for nasal intubation endoscopes is generally smaller than the number of pixels of imagers for oral intubation endoscopes. Therefore, the quality of images captured by nasal intubation endoscopes tends to be lower than the quality of images captured by oral intubation endoscopes.

When the same object is observed, it is preferred that what is observed (i.e. how the object appears in the observation) be similar whether the endoscope used for observation is an oral intubation endoscope or a nasal intubation endoscope. The depth of field is one of the conditions that affects the similarity of what is observed. Therefore, it is preferred that oral intubation endoscopes and nasal intubation endoscopes have a similar depth of field.

The depth of field depends on the focal length and the F-number of the objective optical system and the permissible circle of confusion of the imager. As described above, the number of pixels of imagers used in nasal intubation endoscopes is generally smaller than the number of pixels of imagers used in oral intubation endoscopes. Hence, the focal length of oral intubation endoscopes is larger accordingly. If the pixel size is the same, the permissible circle of confusion is the same. Therefore, to make the depth of field similar, it is necessary to make the F-number of the objective optical systems of oral intubation endoscopes larger.

The brightness is another condition that affects the similarity of what is observed. As described above, the F-number of the objective optical systems of oral intubation endoscopes is larger than the F-number of the objective optical systems of nasal intubation endoscopes. Hence, it is necessary that the light guide exit surface be larger in oral intubation endoscopes than in nasal intubation endoscopes.

The outer diameter of the insert part, the size of the image output area, and the size of the light guide exit surface of an endoscope are interrelated. The following ratios fall within certain respective ranges.
(A) the ratio of the outer diameter of the insert part of the endoscope and the size of the image output area.
(B) the ratio of the outer diameter of the insert part of the endoscope and the size of the light guide exit surface.
(C) the ratio of the size of the image output area and the size of the light guide exit surface.

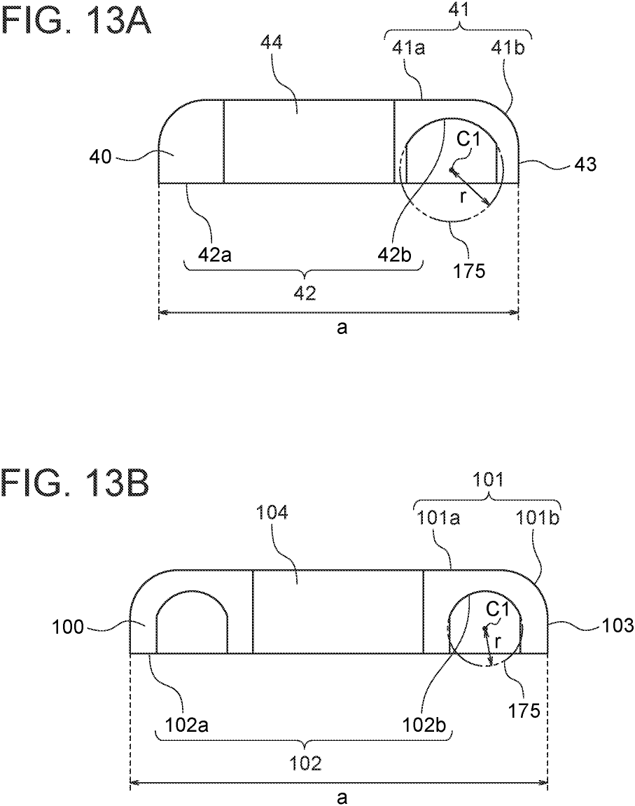

The parameters used in conditional expression (9) will be described below with reference to FIGS. 13.

FIGS. 13A and 13B are cross sectional views of end covers . FIG. 13A illustrates a first example of the end cover. The components that are the same as those in FIG. 2B are denoted by the same reference numerals and will not be described further . FIG. 13B illustrates a second example of the end cover. The components that are the same as those in FIG. 4B are denoted by the same reference numerals and will not be described further.

The end cover 40 shown in FIG. 13A has one illumination area 42b provided on one side of the through bore 44. The end cover 100 shown in FIG. 13B has two illumination areas 102b provided on two opposite sides of the through bore 104.

In FIG. 13A, length a is the outer diameter of the end cover 40. In FIG. 13B, length a is the outer diameter of the end cover 100. The circle 175 is the small circle of the torus surface. Length r is the radius of the small circle.

The side surface of the end cover 40, 100 may be shaped as the side surface of a cylinder. Then, the outer diameter of the end cover 40, 100 is the diameter of the bottom surface of the cylinder.

If the value of a/r exceeds the upper bound of conditional expression (9), the outer diameter of the end cover is too large, or the radius of the small circle is too small.

If the outer diameter of the end cover is too large, the ease of insertion will be deteriorated.

If the radius of the small circle is too small, the amount of illumination light will be insufficient, as described in the above description of the technical meaning of conditional expression (1).

If the value of a/r falls below the lower bound of conditional expression (9), the outer diameter of the end cover is too small, or the radius of the small circle is too large.

If the outer diameter of the end cover is too small, the area of the light guide exit surface is so narrow that the amount of illumination light is insufficient, or the distribution of illumination light is narrow.

Moreover, the diameter of the imaging unit is necessitated to be too small. Then, the image output area of the imager is too small, and the number of pixels is too small. In consequence, it is difficult to produce high quality images.

If the radius of the small circle is too large, the distribution of illumination light is too narrow, as described in the description of the technical meaning of conditional expression (1).

An endoscope system according to this embodiment includes the endoscope according to this embodiment and an image processing apparatus.

The endoscope system according to this embodiment can provide illumination with a wide distribution of illumination light, small loss of illumination light, and small unevenness of illumination.

The endoscope system according to this embodiment can provide illumination with a wide distribution of illumination light, small loss of illumination light, and small unevenness of illumination. In consequence, the endoscope can produce images with low noise and small unevenness in the brightness. Therefore, the endoscope system according to this embodiment can keep high image quality even after applying image processing.

In the following examples of the endoscope will be described with reference to drawings. It should be understood that the invention disclosed herein is not limited by the examples described in the following.

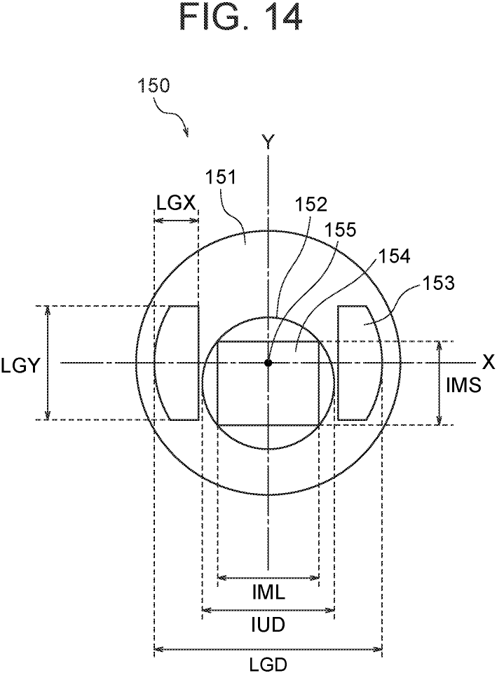

FIG. 14 is a diagram illustrating the end portion of an endoscope as seen from the object side. The components in FIG. 14 that are the same as those in FIG. 7B are denoted by the same reference numerals and will not be described further.

The first to eighth examples of the endoscope described in the following have two light guide exit surfaces 153 disposed on opposite sides of the imaging unit 152. In all the examples, the shape of the image output area 154 is rectangular. The shape of the outer edge of each light guide exit surface 153 is an arc of a circle having its center located on the center axis 155.

In FIG. 14, IUD is the outer diameter of the imaging unit 152, LGD is the diameter of the circle of the outer edges of the light guide exit surfaces 153, LGY is the length of each light guide exit surface 153 in the cross section perpendicular to the second specific cross section, IML is the length of the long side of the image output area 154, and IMS is the length of the short side of the image output area 154.

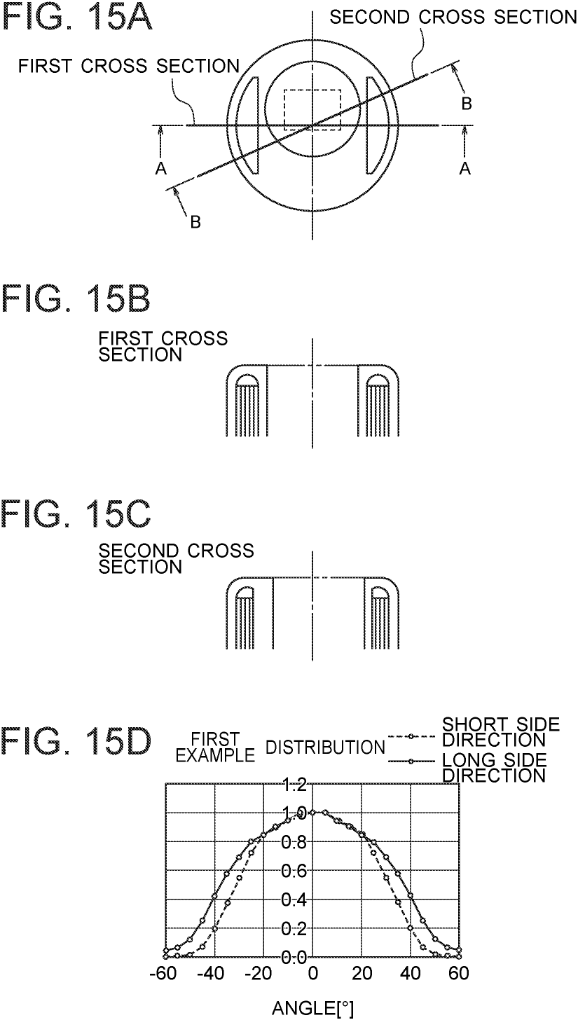

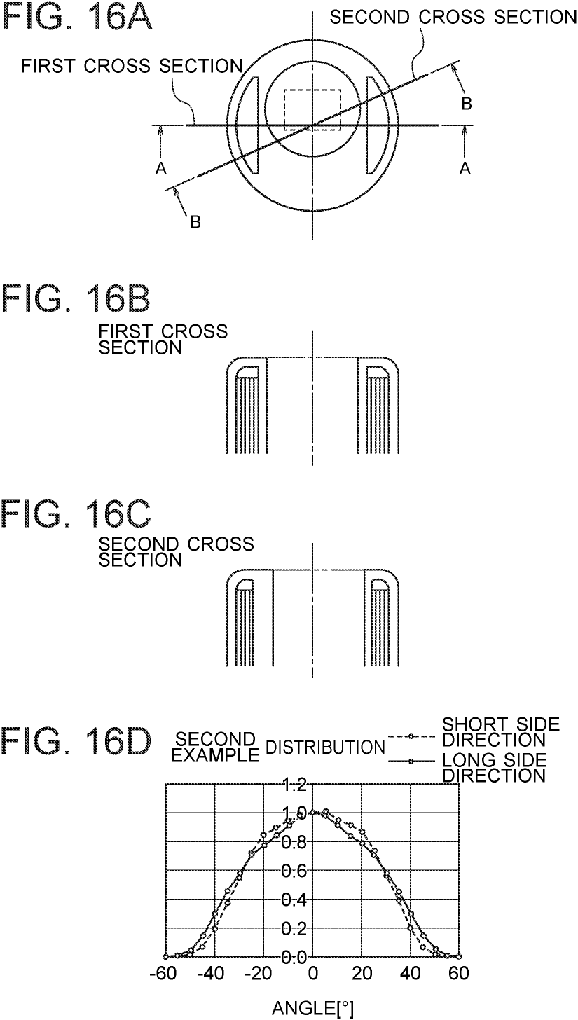

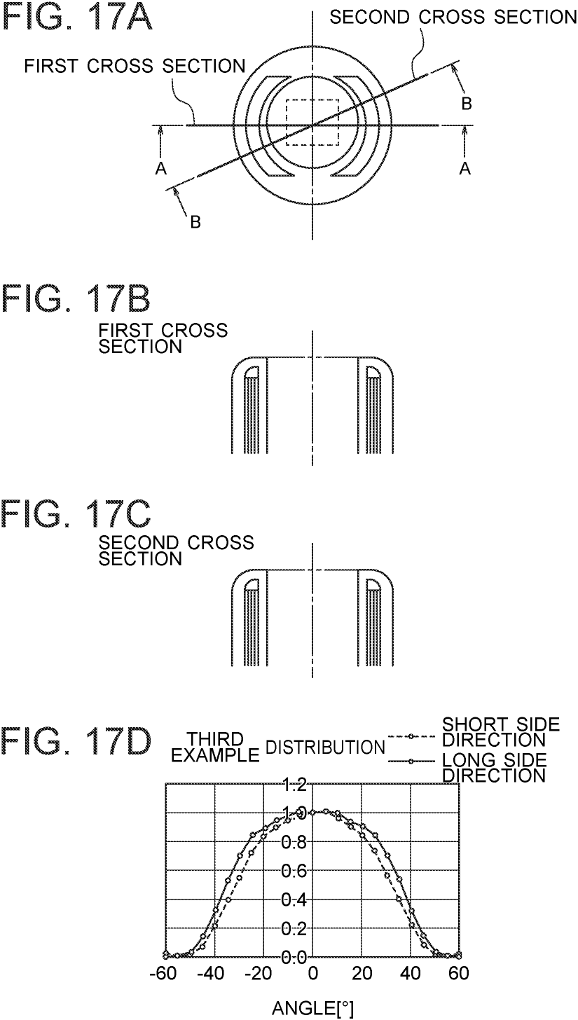

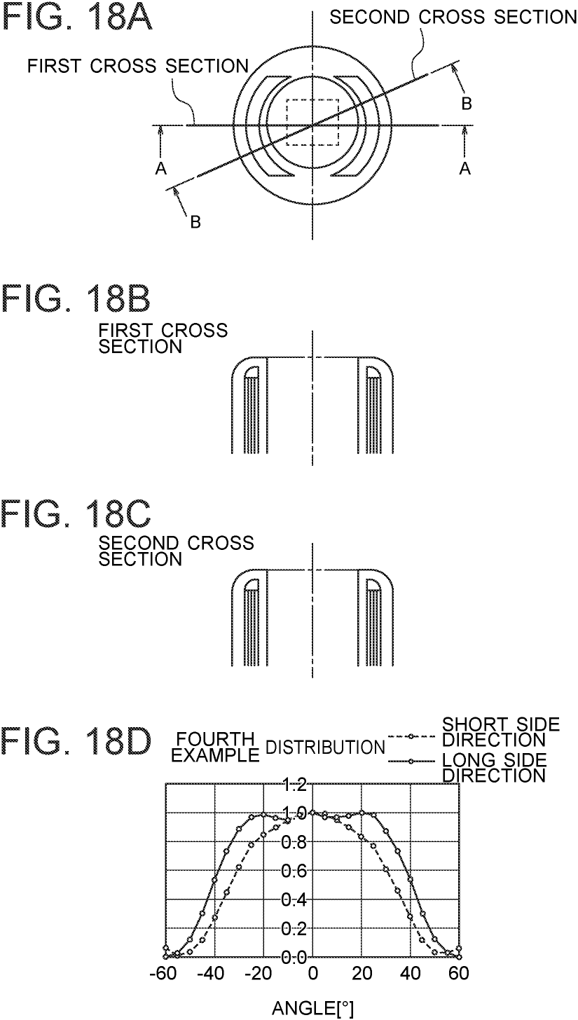

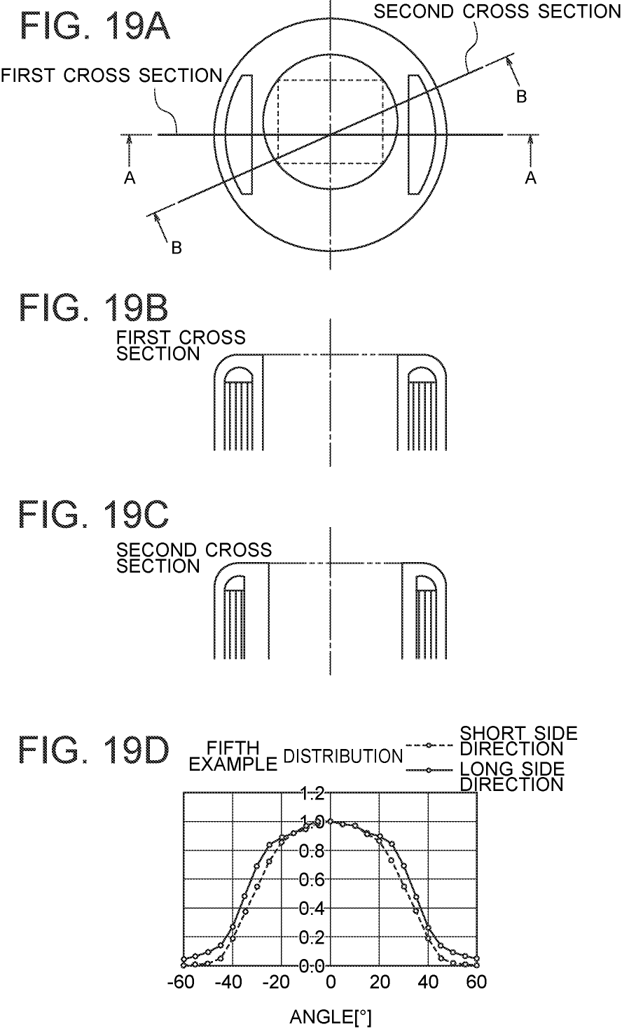

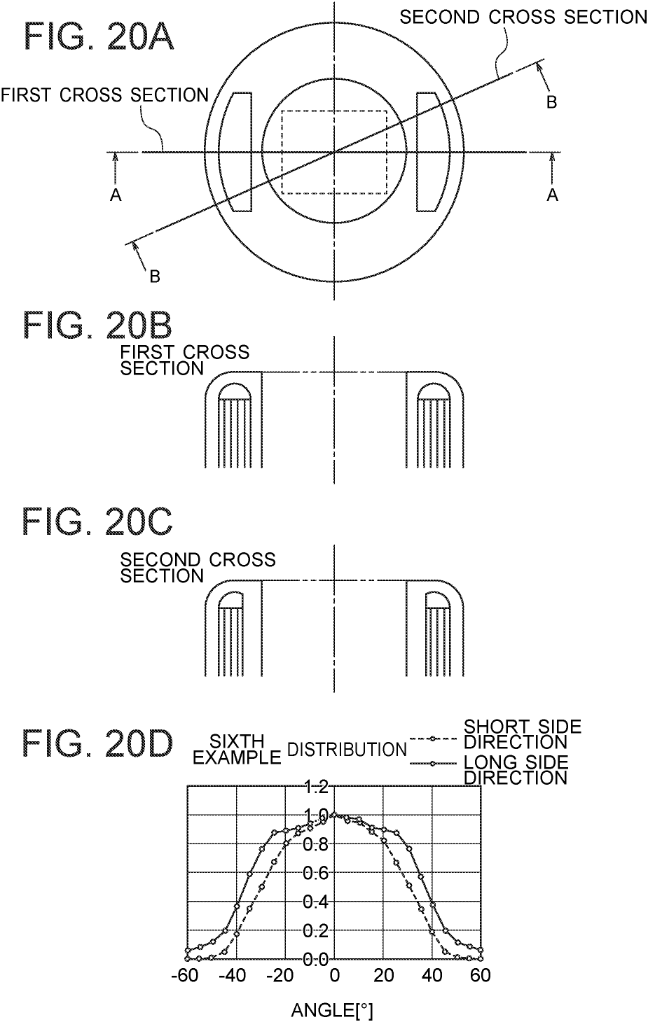

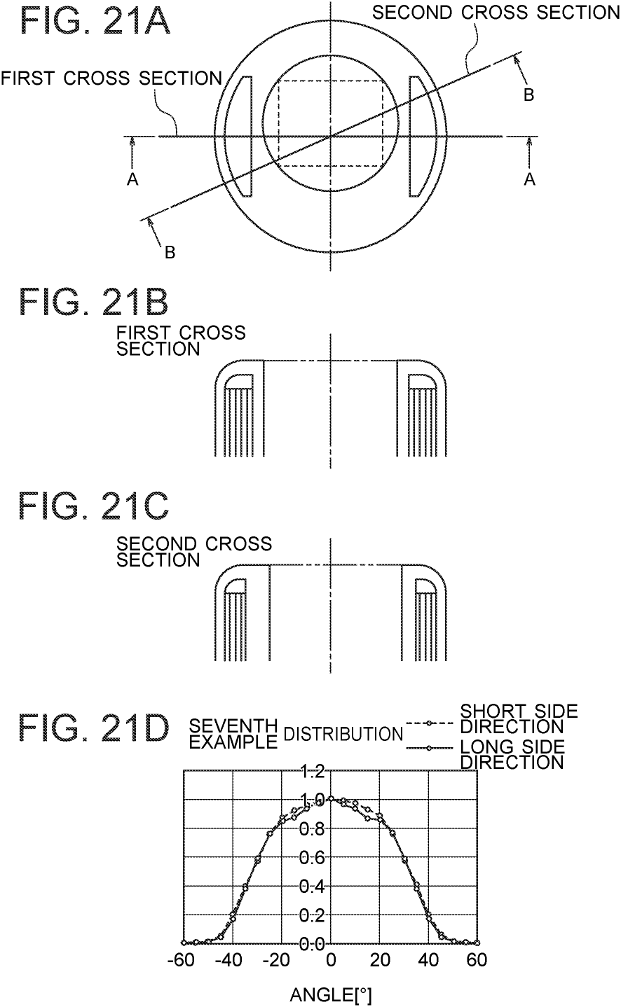

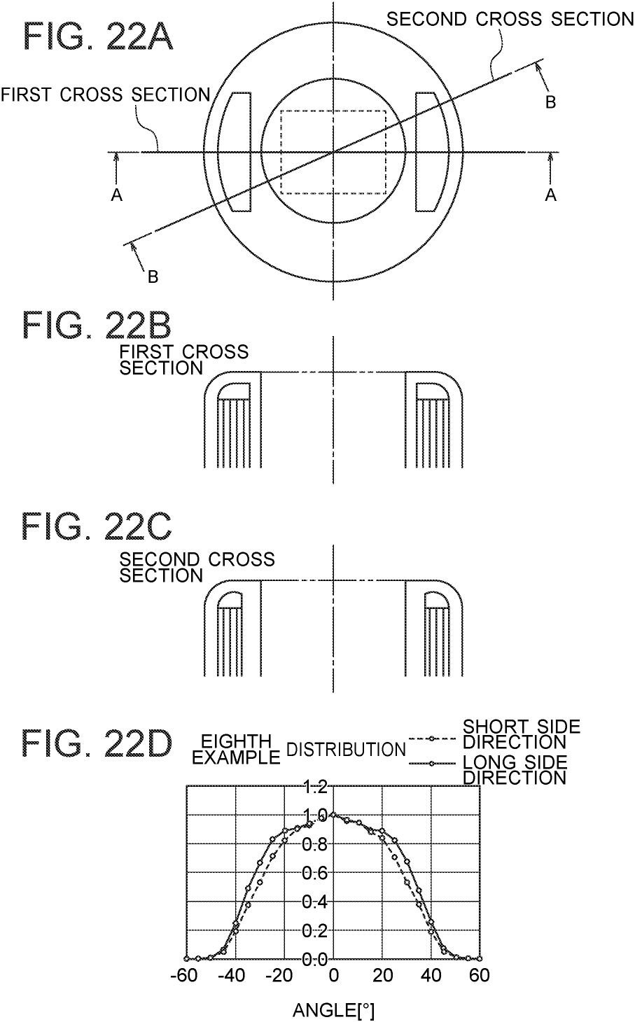

Referring to the drawings showing the examples, FIGS. 15A, 16A, 17A, 18A, 19A, 20A, 21A, and 22A are diagrams showing the end portion of the endoscope as seen from the object side. FIGS. 15B, 16B, 17B, 18B, 19B, 20B, 21B, and 22B are cross sectional views of the end portion of the insert part taken along line A-A. FIGS. 15C, 16C, 17C, 18C, 19C, 20C, 21C, and 22C are cross sectional views of the end portion of the insert part taken along line B-B. FIGS. 15D, 16D, 17D, 18D, 19D, 20D, 21D, and 22D are graphs showing the light distribution of illumination light.

In these drawings, line A-A indicates the location of the first cross section, and line B-B indicates the location of the second cross section.

The first example of the endoscope includes an imaging unit and light guides provided in its insert part. The insert part is provided with an end cover at its end.

The illumination area is formed by a PT surface in its entirety. In the first cross section, the PT surface has a an essentially hemispherical shape. The shape of the light guide exit surface is a rectangle one side of which is replaced by an arc.

The second example of the endoscope includes an imaging unit and light guides provided in its insert part. The insert part is provided with an end cover at its end.

The illumination area is formed by a PT surface and a flat surface. The flat surface is located closer to the center axis than the PT surface. In the first cross section, the shape of the PT surface is essentially a sector. The shape of the light guide exit surface is a rectangle one side of which is replaced by an arc.

The third example of the endoscope includes an imaging unit and light guides provided in its insert part. The insert part is provided with an end cover at its end.

The illumination area is formed by a PT surface in its entirety. In the first cross section, the PT surface has an essentially hemispherical shape. The shape of the light guide exit surface is essentially an annular sector.

The fourth example of the endoscope includes an imaging unit and light guides provided in its insert part. The insert part is provided with an end cover at its end.

The illumination area is formed by a PT surface and a flat surface. The flat surface is located closer to the center axis than the PT surface. In the first cross section, the shape of the PT surface is essentially a sector. The shape of the light guide exit surface is essentially an annular sector.

The fifth example of the endoscope includes an imaging unit and light guides provided in its insert part. The insert part is provided with an end cover at its end.

The illumination area is formed by a PT surface in its entirety. In the first cross section, the shape of the PT surface is essentially a sector. The shape of the light guide exit surface is a rectangle one side of which is replaced by an arc.

The sixth example of the endoscope includes an imaging unit and light guides provided in its insert part. The insert part is provided with an end cover at its end.

The illumination area is formed by a PT surface in its entirety. In the first cross section, the PT surface has an essentially hemispherical shape. The shape of the light guide exit surface is a rectangle one side of which is replaced by an arc.

The seventh example of the endoscope includes an imaging unit and light guides provided in its insert part. The insert part is provided with an end cover at its end.

The illumination area is formed by a PT surface and a flat surface. The flat surface is located closer to the center axis than the PT surface. In the first cross section, the shape of the PT surface is essentially a sector. The shape of the light guide exit surface is a rectangle one side of which is replaced by an arc.

The eighth example of the endoscope includes an imaging unit and light guides provided in its insert part. The insert part is provided with an end cover at its end.

The illumination area is formed by a PT surface and a flat surface. The flat surface is located closer to the center axis than the PT surface. In the first cross section, the shape of the PT surface is essentially a sector. The shape of the light guide exit surface is a rectangle one side of which is replaced by an arc.

Numerical values of the above each of examples are shown below.

| | Example 1 | Example 2 | Example 3 | Example 4 |
|---|---|---|---|---|
| a | 2.6 | 2.6 | 2.4 | 2.4 |
| R | 0.3 | 0.3 | 0.35 | 0.3 |
| r | 0.165 | 0.165 | 0.2 | 0.165 |
| n | 1.6415 | 1.6415 | 1.6415 | 1.6415 |
| t | 0.15 | 0.15 | 0.15 | 0.15 |
| ψ1 | 0 | 0 | 0 | 0 |
| ψ2 | 24 | 24 | 25 | 25 |
| ε | 35.82 | 35.82 | 40.9 | 40.94 |
| θ | 38.7 | 38.7 | 48 | 47.8 |
| hA | 0.011 | 0.011 | 0.019 | 0.029 |
| dy | 0.015 | 0.015 | 0.025 | 0.04 |
| L | 0.165 | 0.165 | 0.19 | 0.155 |
| IUD | 1.44 | 1.44 | 1.4 | 1.4 |
| LGD | 2.3 | 2.3 | 2.03 | 2.03 |
| LGX | 0.33 | 0.33 | 0.205 | 0.205 |
| LGY | 1.44 | 1.44 | 1.5 | 1.5 |
| IML | 0.84 | 0.84 | 0.772 | 0.772 |
| IMS | 0.606 | 0.606 | 0.67 | 0.67 |
| | example 5 | example 6 | example 7 | example 8 |
| a | 3.5 | 3.9 | 3.5 | 3.9 |
| R | 0.35 | 0.4 | 0.35 | 0.4 |
| r | 0.225 | 0.25 | 0.225 | 0.25 |
| n | 1.6415 | 1.6415 | 1.6415 | 1.6415 |
| t | 0.2 | 0.2 | 0.2 | 0.2 |
| ψ1 | 0 | 0 | 0 | 0 |
| ψ2 | 25 | 25 | 25 | 25 |
| ε | 38.58 | 38.58 | 38.58 | 38.58 |
| θ | 34.2 | 30.5 | 34.2 | 30.5 |
| hA | 0.019 | 0.034 | 0.019 | 0.034 |
| dy | 0.025 | 0.045 | 0.025 | 0.045 |
| L | 0.225 | 0.245 | 0.225 | 0.245 |
| IUD | 2.05 | 2.2 | 2.05 | 2.2 |
| LGD | 3.6 | 3.5 | 3.6 | 3.5 |
| LGX | 0.625 | 0.49 | 0.625 | 0.49 |
| LGY | 1.8 | 1.8 | 1.8 | 1.8 |
| IML | 1.576 | 1.576 | 1.576 | 1.576 |
| IMS | 1.26 | 1.26 | 1.26 | 1.26 |

Numerical values of the conditional expressions of the above each of examples are shown below.

| | Example 1 | Example 2 | Example 3 | Example 4 |
|---|---|---|---|---|
| (1) | 0.07 | 0.07 | 0.10 | 0.18 |
| (2) | 0.00 | 0.00 | 0.00 | 0.00 |
| (3) | 0.67 | 0.67 | 0.61 | 0.61 |
| (4) | 0.08 | 0.08 | 0.12 | 0.22 |
| (5) | -0.02 | -0.02 | 0.00 | -0.02 |
| (6) | 1.08 | 1.08 | 1.17 | 1.17 |
| (7) | 1.00 | 1.00 | 0.95 | 0.94 |
| (8) | 15.76 | 15.76 | 12.00 | 14.55 |
| (9) | 0.18 | 0.18 | 0.26 | 0.47 |
| (1) | 0.08 | 0.14 | 0.08 | 0.14 |
| (2) | 0.00 | 0.00 | 0.00 | 0.00 |
| (3) | 0.65 | 0.65 | 0.65 | 0.65 |
| (4) | 0.12 | 0.18 | 0.12 | 0.18 |
| (5) | -0.12 | -0.08 | -0.12 | -0.08 |
| (6) | 0.89 | 0.79 | 0.89 | 0.79 |
| (7) | 1.00 | 0.98 | 1.00 | 0.98 |
| (8) | 15.56 | 15.60 | 15.56 | 15.60 |
| (9) | 0.23 | 0.37 | 0.23 | 0.37 |

The endoscope according to the embodiment uses light guides to provide illumination. Illumination may be provided by light emitting diodes. In the case where light emitting diodes are used, light guides are not used. In this case, the surface of the light diode package may be regarded as the light guide exit surface according to the embodiment. Alternatively, illumination may be provided by a laser diode(s) and a fluorescent element. In the case where a laser diode(s) and a fluorescent element are used, light guides are not used. In this case the surface of the fluorescent element may be regarded as the light guide exit surface according to the embodiment.

The present invention can be suitably applied to an endoscope desired to be capable of providing illumination with a wide distribution of illumination light, small loss of illumination light, and small unevenness of illumination and an endoscope system including such an endoscope.

The present invention can provide an endoscope and endoscope system capable of providing illumination with a wide distribution of illumination light, small loss of illumination light, and small unevenness of illumination.

What is claimed is:

1. An endoscope comprising:
an insert part;
an imaging unit provided in the insert part;
a light guide having a light guide exit surface provided in the insert part; and
an end cover provided at the distal end of the insert part, wherein
illumination light is emitted from the light guide exit surface,
the end cover has a cut-through portion in which the imaging unit is inserted and fixed,
the end cover has a first surface on its object side, a second surface on its operator side, and a side surface on its outer circumference,
the first surface includes a first flat surface and a curved surface,
the curved surface is located between the first flat surface and the side surface,
the second surface has an illumination area on which the illumination light is incident,
at least a portion of the illumination area is formed as a partial torus surface,
first specific cross sections are defined as cross sections containing the center axis of the end cover and intersecting the light guide exit surface, and
the endoscope satisfies the following conditional expression (1) in at least one first specific cross section:
$$0 \leq \text{hA} / \text{r} \leq 0.5$$
where the partial torus surface is defined as a surface partially cut out from a torus surface,
the torus surface is the surface of a body of revolution formed by rotating a circle in a plane about an axis of rotation that lies in the same plane and does not intersect the circle, the circle is referred to as a small circle,
hA is the distance between a first intersection point and a second intersection point,
the first intersection point is the point of intersection of the straight line passing through the center of the small circle and parallel to the center axis and the light guide exit surface,
the second intersection point is the point of intersection of a ray passing through the boundary of the first flat surface and the curved surface and parallel to the center axis in the space between the light guide exit surface and the illumination area and the light guide exit surface,
in the case where the second intersection point is located between the first intersection point and the side surface, the distance is represented by a value with a plus sign, and
r is the radius of the small circle.

2. An endoscope comprising:
an insert part;
an imaging unit provided in the insert part;
a light guide having a light guide exit surface provided in the insert part; and
an end cover provided at the distal end of the insert part, wherein
illumination light is emitted from the light guide exit surface,
the end cover has a cut-through portion in which the imaging unit is inserted and fixed,
the end cover has a first surface on its object side, a second surface on its operator side, and a side surface on its outer circumference,
the first surface includes a first flat surface and a curved surface,
the curved surface is located between the first flat surface and the side surface,
the second surface has an illumination area on which the illumination light is incident,
at least a portion of the illumination area is formed as a partial torus surface,
first specific cross sections are defined as cross sections containing the center axis of the end cover and intersecting the light guide exit surface, and
the endoscope satisfies the following conditional expression (2) in at least one first specific cross section: section:
$$0 \leq \left(\text{hA} \times {\text{n}}^{2}\right) / \text{r} \leq 1.36$$
where a partial torus surface is defined as a surface partially cut out from a torus surface,
the torus surface is the surface of a body of revolution formed by rotating a circle in a plane about an axis of rotation that lies in the same plane and does not intersect the circle, this circle is referred to as a small circle,
hA is the distance between a first intersection point and a second intersection point,
the first intersection point is the point of intersection of the straight line passing through the center of the small circle and parallel to the center axis and the light guide exit surface,
the second intersection point is the point of intersection of a ray passing through the boundary of the first flat surface and the curved surface and parallel to the center axis in the space between the light guide exit surface and the illumination area and the light guide exit surface,
in the case where the second intersection point is located between the first intersection point and the side surface, the distance is represented by a value with a plus sign, n is the refractive index of the material of the end cover for the e-line, and r is the radius of the small circle.

3. An endoscope according to claim 1, wherein
the imaging unit includes an imager,
the imager has a rectangular image output area within which the subject image can be acquired from the imager,
a first cross section is defined as a cross section expressed by the following equation (3),
a second cross section is defined as a cross section that satisfies the following conditional expression (4),
the first cross section and the second cross section are the first specific cross sections, and
conditional expression (1) is satisfied in the first cross section and the second cross section:
$$\text{φ} 1 = 0$$
$$0.2 \leq \text{φ} 2 / \text{ε} \leq 0.7$$
where φ1 is the angle formed by a second specific cross section and the first cross section,
φ2 is the angle formed by the second specific cross section and the second cross section,
the second specific cross section is the cross section parallel to the long side of the image output area and containing the center axis,
ε is the angle formed by a cross section containing the long side of the image output area and a cross section containing the diagonal of the image output area.

4. An endoscope according to claim 1, wherein the endoscope satisfies the following conditional expression (5):
$$0 < \left(\text{dy} \times \text{n}\right) / \text{R} < 0.5$$
where dy is the distance between the first intersection point and a third intersection point,
in the case where the third intersection point is located between the first intersection point and the side surface, the distance is represented by a value with a plus sign,
the third intersection point is the point of intersection of the straight line passing through the center of curvature of the curved surface in the first surface and parallel to the center axis and the light guide exit surface,
n is the refractive index of the material of the end cover for the e-line, and
R is the curvature radius of the curved surface in the first surface.

5. An endoscope according to claim 4, wherein the endoscope satisfies the following conditional expression (6):
$$- 0.15 < \text{n} \times \left(\text{R} - \text{r} - \text{t}\right) < 0$$
where n is the refractive index of the material of the end cover for the e-line,
R is the curvature radius of the curved surface,
r is the radius of the small circle, and
t is the shortest distance between the first flat surface and the illumination area.

6. An endoscope according to claim 1, wherein the illumination area includes a second flat surface and the partial torus surface, the second flat surface being located closer to the center axis than the partial torus surface.

7. An endoscope according to claim 1, wherein
the imaging unit includes an imager,
the imager has a rectangular image output area within which the subject image can be acquired from the imager,
the outer circumference of the light guide exit surface is composed of an inner edge, an intermediate edge, and an outer edge,
the inner edge being located closer to the center axis than the outer edge,
the intermediate edge being located between the inner edge and the outer edge, the outer edge having the shape of an arc of a circle having its center located on the center axis, and
the endoscope satisfies the following conditional expression (7):
$$0.7 < \text{θ} / \text{ε} < 1.2$$
where θ is the angle formed by a second specific cross section and a third cross section,
the second specific cross section is the cross section containing the center axis and parallel to the long side of the image output area,
the third cross section is the cross section containing the point of intersection of the outer edge and the intermediate edge and the center axis,
ε is the angle formed by a cross section containing the long side of the image output area and a cross section containing the diagonal of the image output area.

8. An endoscope according to claim 1, wherein
the outer circumference of the light guide exit surface is composed of an inner edge, an intermediate edge, and an outer edge, the inner edge being located closer to the center axis than the outer edge,
the intermediate edge being located between the inner edge and the outer edge,
the outer edge having the shape of an arc of a circle having its center located on the center axis, and
the endoscope satisfies the following conditional expression (8) in at least one first specific cross section:
$$0.6 < \text{L} / \text{r} \leq 1.0$$
where L is the distance between the first intersection point and the outer edge, and
r is the diameter of the small circle.

9. An endoscope according to claim 1, wherein the endoscope satisfies the following conditional expression (9):
$$10 < \text{a} / \text{r} < 16$$
where a is the outer diameter of the end cover, and r is the radius of the small circle.

10. An endoscope according to claim 8, wherein the endoscope satisfies the following conditional expression (9):
$$10 < \text{a} / \text{r} < 16$$
where a is the outer diameter of the end cover, and r is the radius of the small circle.

11. An endoscope system comprising an endoscope according to claim 1 and an image processing apparatus.

12. An endoscope system comprising an endoscope according to claim 2 and an image processing apparatus.
